(19) United States
(12) Patent Application Publication
(10) Pub. No.: US 2024/0390157 A1
(43) Pub. Date: Nov. 28, 2024

(54) EXPANDABLE CERVICAL INTERBODY AND INSTRUMENTS THEREOF

(71) Applicant: Warsaw Orthopedic, Inc., Warsaw, IN (US)

(72) Inventors: Stanley Kyle Hayes, Mission Viejo, CA (US); Darren L. Davis, Knoxville, TN (US); Patrick Ryan Keene, Youngsville, NC (US); Cristian A. Capote, Memphis, TN (US); John A. Hall, Germantown, TN (US); Robert M. Loke, Memphis, TN (US)

(21) Appl. No.: 18/665,521

(22) Filed: May 15, 2024

Related U.S. Application Data
(60) Provisional application No. 63/503,859, filed on May 23, 2023.

(51) Int. Cl. A61F 2/44; A61F 2/30; A61F 2/46

(52) U.S. Cl. CPC A61F 2/4425; A61F 2/30734; A61F 2/30771; A61F 2/4611; A61F 2002/30471; A61F 2002/30481; A61F 2002/30528; A61F 2002/30579; A61F 2002/30736; A61F 2002/30827; A61F 2002/30884; A61F 2002/443; A61F 2002/4622; A61F 2002/4627; A61F 2002/4668

(57) ABSTRACT

A system including an implant and a tool for inserting and expanding the medical implant and locking the implant in place is disclosed. The medical implant may include an expandable body defined by a superior endplate and an inferior endplate that are hingedly coupled and may be expanded and lordosed. The implant may include a wedge disposed between the superior and inferior endplates that is configured to slide across a channel on inferior endplate and along an inclined groove on superior endplate. In at least some embodiments, an expansion screw is operative coupled to the threaded portion of the wedge that when expansion screw is rotated, the wedge is translated thereby expanding the implant.

### CROSS-REFERENCE TO RELATED APPLICATION

This application claims the benefit of and priority to U.S. Provisional Patent Application No. 63/503,859 filed May 23, 2023, the entire disclosure of which is incorporated by reference herein.

In one aspect, the present technology is generally related to an externally driven and expandable implant for use in a medical procedure that may, for example, be related to the spine and more specifically may be useful as an intervertebral implant. In another aspect, the present technology is generally related to a surgical tool for inserting and expanding the externally driven and expandable intervertebral implant. In some embodiments, disclosed implants and surgical tools may form a system that may be used in an anterior cervical discectomy and fusion (ACDF) procedure although other uses in other areas of the spine or between two bones are also contemplated.

### BACKGROUND

Mechanically operated interbody implants may be used to align and/or realign a patient's spine during a medical procedure and/or for purposes of fusion, degenerative tissue and/or trauma/repair procedures. Conventional expandable implants designed for the thoracic and lumbar region of the spine often include top and bottom endplates and a mechanical means to separate the top and bottom endplates. The mechanical mechanisms to separate the top and bottom endplates are often cumbersome and require a large footprint that is often unsuitable, for example, for ACDF type surgeries of the cervical portion of the spine. Many currently available ACDF type implants may be limited in their ability to optimize the adjustment of lordosis or sagittal alignment of the vertebral bodies because they may rely on a fixed lordotic angle between the superior/cephalad and inferior/caudad faces of the device.

### SUMMARY

The techniques of this disclosure generally relate to an expandable implant including a superior endplate and an inferior endplate hingedly coupled and which may further include a locking element to secure the inferior endplate and superior endplate in a particular configuration, for example. The superior and inferior endplates may be moved in a multitude of expanded and/or lordosed or kyphosed or otherwise angled configurations via an external surgical tool for example.

In one aspect, the present disclosure provides for an expandable implant movable between a contracted position (closed position) and an expanded position, for example. The expandable implant may include an expandable body extending from a proximal end to a distal end in a proximal-to-distal direction (may also be referred to as an anterior-to-posterior direction depending on surgical technique), extending from a first lateral side to a second lateral side in a widthwise direction, and extend from a superior endplate to an inferior endplate in a heightwise direction (may also be referred to as a cephalad-to-caudal and/or vertical direction depending on surgical technique), for example. In various embodiments, the expandable body may be defined by a superior endplate and an inferior endplate that are hingedly connected, for example. In various embodiments, the inferior endplate may include a support frame having a proximal surface and a distal surface, a wedge channel, and a hinge member. In some embodiments, the superior endplate may include an arcuate channel, a ramp having an inclined surface coaxially in line with a support frame, and a wedge pin groove. In some embodiments, the expandable implant may comprise a drive wedge disposed between the superior endplate and the inferior endplate having a bearing surface on an upper end thereof configured to slide across the inclined ramp and a tang on a lower end thereof configured for disposal within the wedge channel. In various embodiments, the implant may include an expansion screw extending through the support frame and being operatively coupled to the wedge so that when the expansion screw is rotated, the wedge is linearly translated in a longitudinal direction thereby expanding the implant.

In another aspect, the disclosure provides for a system including a medical implant and a surgical tool, for example. The system may include an expandable implant movable between a contracted position and an expanded position, for example. In various embodiments, the expanded position may also refer to a distracted and angled orientation of the superior endplate and inferior endplate. The expandable implant may include an expandable body extending from a proximal end to a distal end in a proximal-to-distal direction and extending from a first lateral side to a second lateral side in a widthwise direction, for example. In various embodiments, the expandable body may be defined by a superior endplate and an inferior endplate that are hingedly connected, for example. In various embodiments, the inferior endplate may include a support frame having a proximal surface and a distal surface, a wedge channel, and a hinge member. In some embodiments, the superior endplate may include an arcuate channel, a ramp having an inclined surface coaxially in line with a support frame, and a wedge pin groove. In some embodiments, the expandable implant may comprise a drive wedge disposed between the superior endplate and the inferior endplate having a bearing surface on an upper end thereof configured to slide across the inclined ramp and a tang on a lower end thereof configured for disposal within the wedge channel. In various embodiments, the implant may include an expansion screw extending through the support frame and being operatively coupled to the wedge so that when the expansion screw is rotated, the wedge is linearly translated in a longitudinal direction thereby expanding the implant. In various embodiments, the system may also include a surgical tool for inserting and expanding the implant and tightening the expansion screw while the implant is expanded at a desired height, position, and/or angle. In some embodiments, the surgical tool may include a plunger having a mounting jaw extending in a lengthwise direction from a proximal end thereof to a distal end thereof that engages with the gripping grooves of disclosed expandable implants to insert, and release disclosed implants. In various embodiments, the surgical tool may include a drive shaft having a drive end disposed within hollow aperture of the plunger to rotate the expansion screw and expand the implant at a desired height, position and/or angle. Furthermore, in various embodiments, the drive end may have a size and shape generally corresponding to a size and shape of a drive feature of the expansion screw. In some embodiments, the mounting jaw may have a size and shape generally corresponding to the shape of the gripping grooves of the expandable implant. In various embodiments, the surgical tool may include an angle indicator ring to identify the angle of inclination of the expanded implant.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

FIG. 1 is a perspective view of an expandable implant.

FIG. 2 is a top-down view of an expandable implant.

FIG. 3 is an alternative perspective view of an expandable implant.

FIG. 4 is an exploded parts view of an expandable implant.

FIG. 5 is a perspective view of the interior of a superior endplate of an expandable implant.

FIG. 6 is a perspective cross section view of an expandable implant FIG. 7 is a perspective parts view of a second expandable implant.

FIG. 8 is a perspective parts view of a third expandable implant

FIG. 9 is a side view of an expandable implant embodiment in a contracted position.

FIG. 10 is a side view of an expandable implant embodiment in an open position.

FIG. 11 is a top-down view of an expandable implant.

FIG. 12 is an exploded parts view of an expandable implant.

FIG. 13 is a perspective view of the interior of a superior endplate of an expandable implant.

FIG. 14 is a perspective view of the interior of an inferior endplate of an expandable implant.

FIG. 15 is a side view of an expandable implant showing a bone screw trajectory.

FIG. 16 is a front view of an expandable implant.

FIG. 17 is a front view of an enlarged area of FIG. 16.

FIG. 18 is a side view of an expandable implant inserted between vertebrae.

FIG. 19 is a front view of an expandable implant inserted between vertebrae.

FIG. 20 is a perspective view of an expandable implant inserted between vertebrae.

FIG. 21 is a perspective view of a surgical tool for use with disclosed expandable implants.

FIG. 22 is a top-down view of a surgical tool for use with disclosed expandable implants.

FIG. 23 is a side down view of a surgical tool for use with disclosed expandable implants.

FIG. 24 is an exploded parts view of a surgical tool for use with disclosed implant embodiments.

FIG. 25A is a cross section view showing a distal end of a surgical tool coupled with a disclosed implant embodiment.

FIG. 25B is a cross section view showing a proximal end of a surgical tool coupled with a disclosed implant embodiment.

FIG. 26A is a perspective view showing a distal end of a surgical tool coupled with a disclosed implant embodiment.

FIG. 26B is a perspective view showing the proximal end of a surgical tool coupled with a disclosed implant embodiment.

FIG. 27A is a side perspective of a distal end of a surgical tool coupled with a cross section view showing disclosed implant embodiment in a contracted position.

FIG. 27B is a side perspective of a proximal end of a surgical tool coupled with a disclosed implant embodiment in a contracted position.

FIG. 28A is a side perspective of a distal end of a surgical tool coupled with a cross section view showing disclosed implant embodiment in an expanded position.

FIG. 28B is a side perspective of a proximal end of a surgical tool coupled with a disclosed implant embodiment in an expanded position.

FIG. 29 is a reference drawing showing the human spine of which various disclosed implant embodiments may be installed in.

FIG. 30 is a reference drawing showing various planes and reference directions of which the various disclosed implant embodiments may move in or act in with respect to a patient.

### DETAILED DESCRIPTION

Embodiments of the present disclosure relate generally, for example, to spinal stabilization systems, and more particularly, to surgical instruments for use with spinal stabilization systems. Embodiments of the devices and methods are described below with reference to the Figures.

The following discussion omits or only briefly describes certain components, features and functionality related to medical implants, installation tools, and associated surgical techniques, which are apparent to those of ordinary skill in the art. It is noted that various embodiments are described in detail with reference to the drawings, in which like reference numerals represent like parts and assemblies throughout the several views, where possible. Reference to various embodiments does not limit the scope of the claims appended hereto because the embodiments are examples of the inventive concepts described herein. Additionally, any example(s) set forth in this specification are intended to be non-limiting and set forth some of the many possible embodiments applicable to the appended claims. Further, particular features described herein can be used in combination with other described features in each of the various possible combinations and permutations unless the context or other statements clearly indicate otherwise.

Terms such as “same,” “equal,” “parallel,” “perpendicular,” etc. as used herein are intended to encompass a meaning of exactly the same while also including variations that may occur, for example, due to manufacturing processes. The term “substantially” may be used herein to emphasize this meaning, particularly when the described embodiment has the same or nearly the same functionality or characteristic, unless the context or other statements clearly indicate otherwise. The term “about” may also be used herein to emphasize this meaning and if a value and/or a range of values is provided in the specification or claims with the modifier “about” a meaning of +/− ten percent (10%) to those provided values are encompassed by the meaning of “about,” unless the context clearly indicates otherwise.

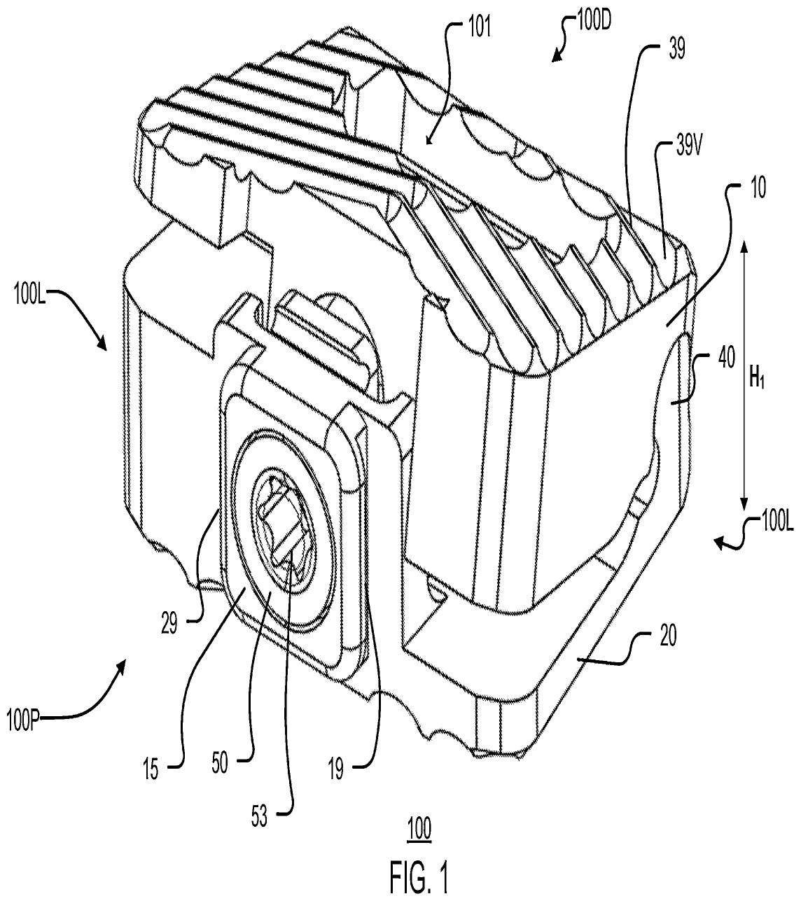

Referring to FIGS. 1-28B generally, various embodiments and views of an expandable implant 100 and corresponding surgical tool 200 are disclosed. The components of expandable implant 100 and surgical tool 200 can be fabricated from biologically acceptable materials suitable for medical applications, including metals, synthetic polymers, ceramics, and bone material and/or their composites. For example, the components, individually or collectively, can be fabricated from materials such as stainless steel alloys, commercially pure titanium, titanium alloys, Grade 5 titanium, super-clastic titanium alloys, cobalt-chrome alloys, superelastic metallic alloys (e.g., Nitinol, super elasto-plastic metals, such as GUM METAL™), ceramics and composites thereof such as calcium phosphate (e.g., SKELITE™), thermoplastics such as polyaryletherketone (PAEK) including polyetheretherketone (PEEK), polyetherketoneketone (PEKK) and polyetherketone (PEK), carbon-PEEK composites, PEEK-BaSO4 polymeric rubbers, polyethylene terephthalate (PET), fabric, silicone, polyurethane, silicone-polyurethane copolymers, polymeric rubbers, polyolefin rubbers, hydrogels, semi-rigid and rigid materials, elastomers, rubbers, thermoplastic elastomers, thermoset elastomers, elastomeric composites, rigid polymers including polyphenylene, polyamide, polyimide, polyetherimide, polyethylene, epoxy, bone material including autograft, allograft, xenograft or transgenic cortical and/or corticocancellous bone, and tissue growth or differentiation factors, partially resorbable materials, such as, for example, composites of metals and calcium-based ceramics, composites of PEEK and calcium based ceramics, composites of PEEK with resorbable polymers, totally resorbable materials, such as, for example, calcium based ceramics such as calcium phosphate, tri-calcium phosphate (TCP), hydroxyapatite (HA)-TCP, calcium sulfate, or other resorbable polymers such as polyactide, polyglycolide, polytyrosine carbonate, polycaroplactohe, polylactic acid or polylactide and their combinations.

In various embodiments, components may be coated with a ceramic, titanium, and/or other biocompatible material to provide surface texturing at (a) the macro scale, (b) the micro scale, and/or (c) the nano scale, for example. Similarly, components may undergo a subtractive manufacturing process providing for surface texturing configured to facilitate osscointegration and cellular attachment, and osteoblast maturation. Example surface texturing of additive and subtractive manufacturing processes may comprise (a) macro-scale structural features having a maximum peak-to-valley height of about 40 microns to about 500 microns, (b) micro-scale structural features having a maximum peak-to-valley height of about 2 microns to about 40 microns, and/or (c) nano-scale structural features having a maximum peak-to-valley height of about 0.05 microns to about 5 microns. In various embodiments, the three types of structural features may be overlapping with one another, for example. Additionally, such surface texturing may be applied to any surface, e.g., both external exposed facing surfaces of components and internal nonexposed surfaces of components. Further discussion regarding relevant surface texturing and coatings is described in, for example, U.S. Pat. No. 11,096,796, titled Interbody spinal implant having a roughened surface topography on one or more internal surfaces, and filed on Mar. 4, 2013—the entire disclosure of which is incorporated herein by reference in its entirety. Accordingly, it shall be understood that any of the described coating and texturing processes of U.S. Pat. No. 11,096,796, may be applied to any component of the various embodiments disclosed herein, e.g., the exposed surfaces and internal surfaces of endplates. Another example technique for manufacturing an orthopedic implant having surfaces with osteoinducting roughness features including micro-scale structures and nano-scale structures is disclosed in U.S. Pat. No. 10,821,000, the entire contents of which are incorporated herein by reference. Additionally, an example of a commercially available product may be the Adaptix™ Interbody System sold by Medtronic and comprising a titanium cage made with Titan nanoLOCK™.

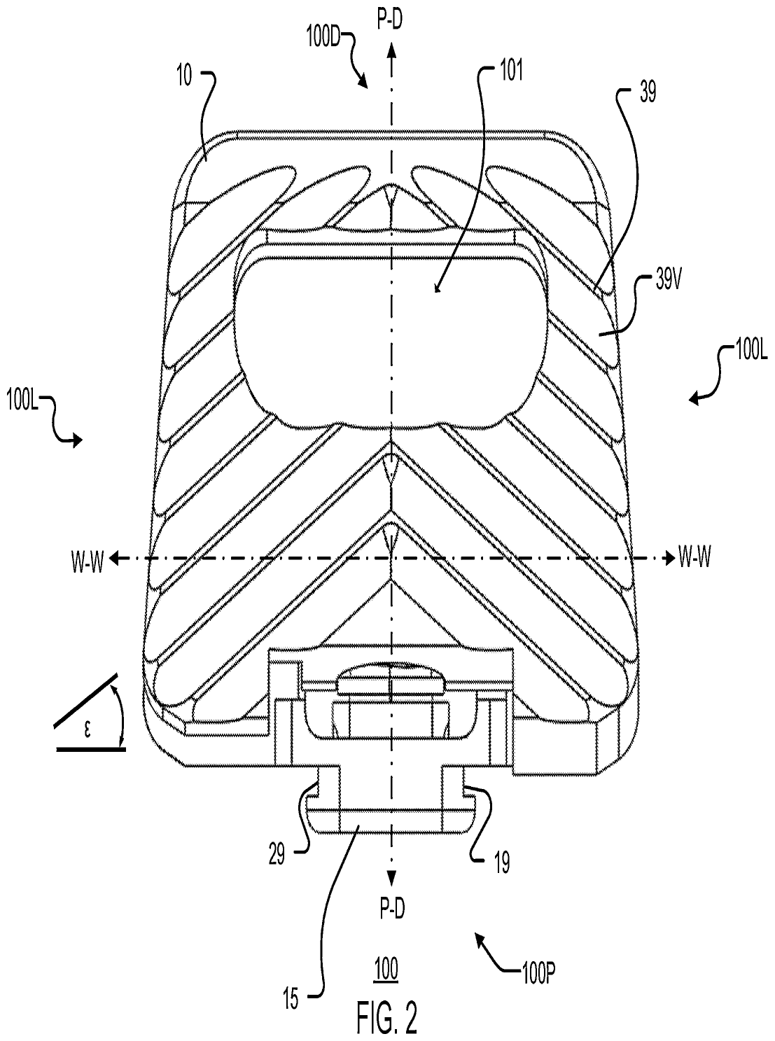

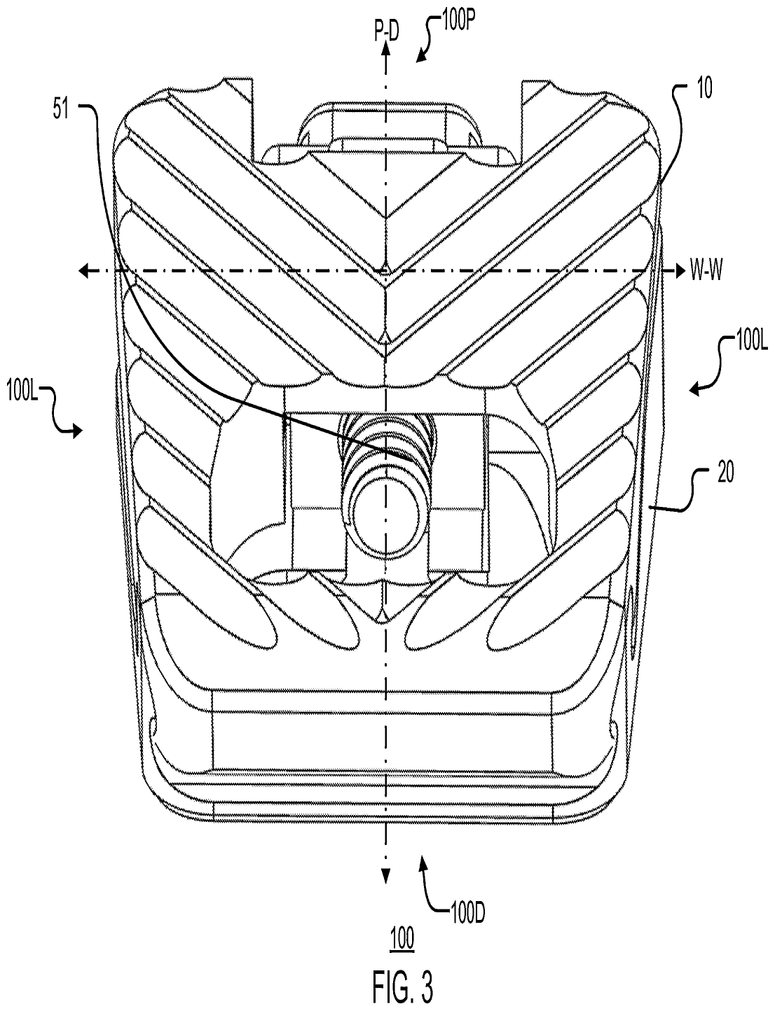

Referring generally to FIGS. 1-3 various views of an expandable implant, or implant 100 in a collapsed position are illustrated. FIG. 1 is a perspective view of an expandable implant 100 having a first height H₁ or thickness between the superior endplate 10 and inferior endplate 20. FIG. 2 is a top-down view of an expandable implant 100. FIG. 3. Is another perspective of an expandable implant 100. In the example embodiment, expandable implant 100 may include a proximal end 100P, a distal end 100D, and lateral sides 100L.

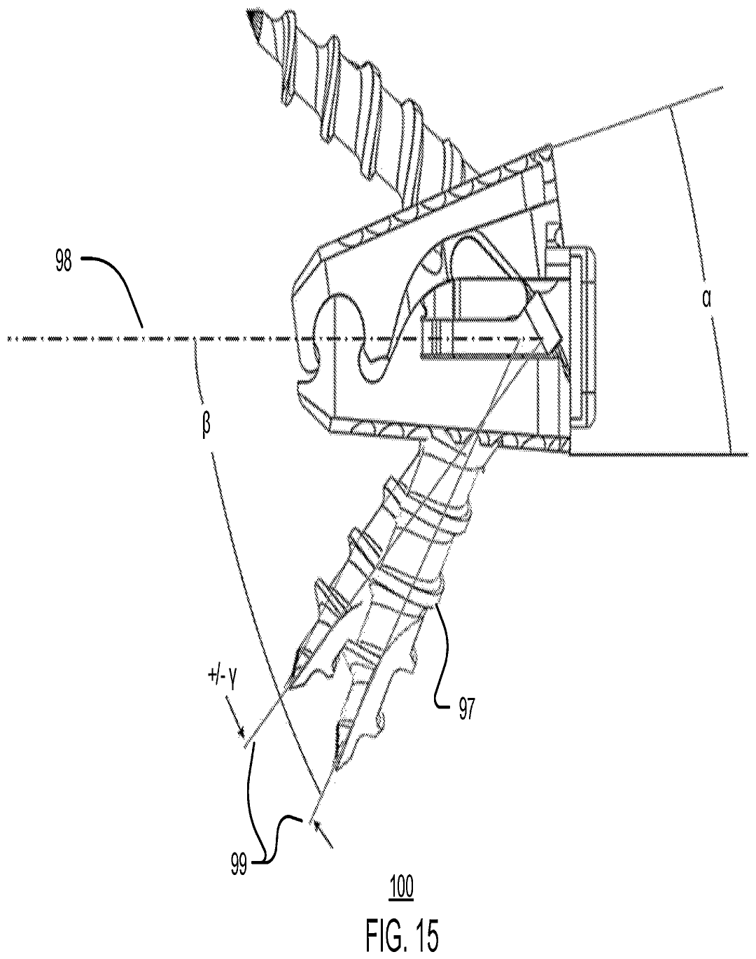

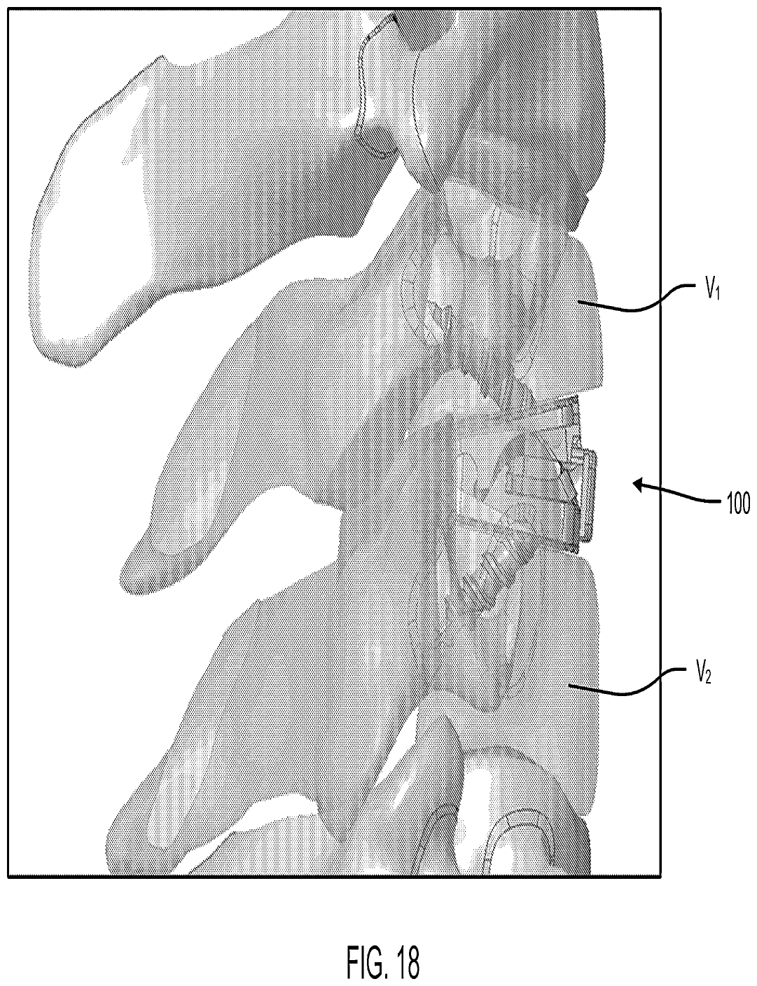

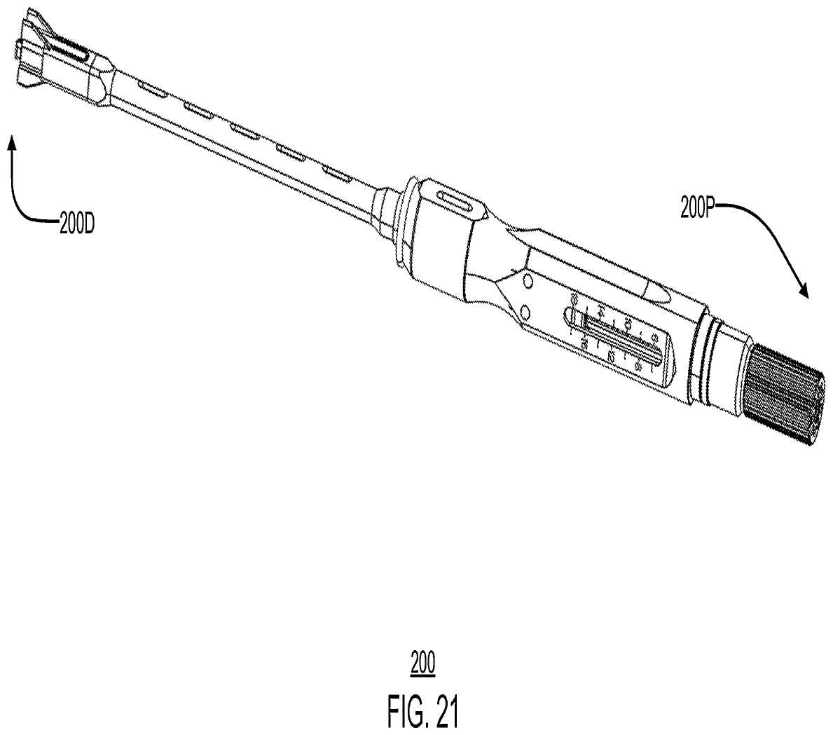

In various embodiments, and as illustrated in FIGS. 1-2, gripping grooves 19, 29 may be present at the proximal end 100P of implant 100 for coupling with corresponding prongs of a surgical tool 200 (see FIGS. 21-28) disclosed herein. In various embodiments, the number of gripping grooves may vary, e.g., gripping groves may be 2, 3, 4, or more. In various embodiments, implant 100 may be referred to as an externally driven expandable implant because an end user or surgeon may use a surgical tool to open and close implant, e.g., expandable implant 100. A surgical tool 200 may adjust the lordotic angle of implant 100 by actuation of an expansion mechanism as will be explained in detail with respect to FIGS. 21-28. Implant 100 may be expanded to a patient specific and appropriate lordotic angle α, as seen in FIG. 15 (also referred to as angle of inclination) by actuation of the expansion mechanism. Thereafter, a surgeon may lock the implant 100 in place by securing bone screws 97 into the adjacent vertebra V₁ and V₂ (see FIGS. 18-20). In some embodiments, superior endplate 10 may be referred to as a “cephalad” endplate, and inferior endplate 20 may be referred to as a “caudal” endplate consistent with standard medical terminology, e.g., particularly for an ACDF implant region of interest.

In some embodiments, implant 100 may include angled engagement features 39. In the example embodiment, engagement features 39 extend diagonally across the exposed uppermost surface of superior endplate 10 and across the exposed lowermost surface of inferior endplate 20. The engagement features 39 comprise flattened top rails that are sequentially spaced apart with rounded bottom valleys 39V therebetween. As seen best in the top-down view of FIG. 2, the engagement features 39 of the superior endplate 10 are oriented at an angle ε with respect to a proximal face of the superior endplate 10. Similarly, the engagement features 39 of the inferior endplate 20 are oriented at an angle ε with respect to a proximal face of the inferior endplate 20. In various embodiments, the angle ε may be about 20 to about 40 degrees and in at least one embodiment the angle ε may be angled at about 30 degrees. At least one advantage of this orientation may be a relatively greater resistance and/or suppression of expulsion of the implant 100 relative to embodiments in which engagement features solely extend horizontally across the implant 100. In the example embodiment, by orienting the engagement features 39 diagonally, the implant 100 may resist expulsion in multiple directions, e.g., forward flexion/extension and lateral bending. In the example embodiment, it may be seen that the engagement features 39 take a “V” shape pattern across the respective outer surface of the superior endplate 10 and inferior endplate 10. Additionally, it may be seen that an apex of the engagement features 39, where a change in direction occurs, is substantially centered along a middle axis that bisects implant 100 in the proximal-to-distal direction P-D.

At least one advantage of relying on an external tool to adjust a lordotic angle of implant 100 may be the case in expansion and the setting of the implant at a particular lordotic angle by a corresponding surgical tool 200 with a calibrated indicator. Expansion of the implant 100 with a moving mechanism may also be gentler on the patient anatomy due to a relatively consistent and controlled expansion versus other tools relying on jaws, forceps, or reverse pliers. Optimization of the footprint of the expansion mechanism is desirable to ensure that implant 100 may have a relatively large void space in the interior thereof, which may facilitate a fusion process during an ACDF procedure. For example, implant 100 may have a relatively large internal volume 101 that is open through the superior endplate 10 and inferior endplate 20 which may be packed with bone graft material, for example. In this vein, a moving direction of a wedge and expansion mechanism should be optimized to keep the internal volume 101 as large as reasonably possible.

As illustrated in FIGS. 2-3, implant 100 may extend in a proximal-to-distal direction from the proximal end 100P to the distal end 100D. In the art, the proximal-to-distal direction may also be referred to as a longitudinal direction and/or an anterior-to-posterior direction depending on surgical technique and final orientation. Similarly, the proximal end 100P may be referred to as an anterior end depending on surgical technique and final orientation and the distal end 100D may be referred to as a posterior end depending on surgical technique and final orientation. As illustrated, the proximal-to-distal direction may extend parallel to axis P-D through the center of the implant 100, for example. Implant 100 may extend in a widthwise direction (also referred to as lateral direction or transverse direction) from the first lateral side 100L to the second lateral side 100L through axis W-W through the center of implant 100 and the center of expansion screw 50, for example. The axis P-D may be perpendicular and/or substantially perpendicular to the axis W-W. For example, the proximal-to-distal direction may be perpendicular to the widthwise direction. Additionally, the width of the implant may taper from a proximal end 100P where it is widest towards a distal end 100D where it is narrowest to better conform to a disc space. In various embodiments, implant 100 may extend from a superior endplate 10 to an inferior endplate 20 in a heightwise direction (may also be referred to as a cephalad-to-caudal direction and/or vertical direction depending on surgical technique and final orientation).

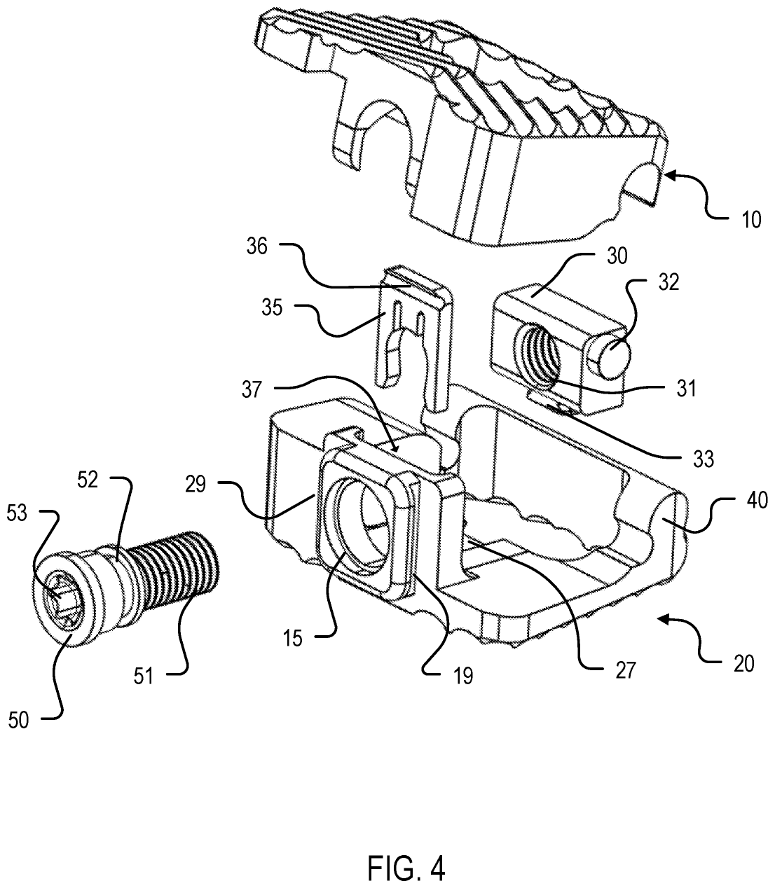

FIG. 4 is a perspective exploded parts view of an expandable implant 100. In the example embodiment, an expansion mechanism including an expansion screw 50 and wedge 30 is illustrated. Expansion screw 50 may include an external thread 51 on an outside circumferential surface thereof, for example. In some embodiments, a recess 37 is formed in the proximal surface of the frame portion 15, and a head portion of the drive expansion screw is seated within the recess 37. In some embodiments, expansion screw 50 may be disposed in recess 37 and may include an expansion screw groove 52 that mates with retaining clip 35 to support the expansion screw 50 in place. For example, this configuration may constrain relative linear movement of the expansion screw 50 only allowing the rotation of the expansion screw 50 thereby proximally translating wedge 30 separating superior endplate 10 from inferior endplate 20. In various embodiments, the retaining clip 35 and the recess 37 are configured to provide friction on the expansion screw to prevent the expansion screw from inadvertently rotating thereby preventing unwanted expansion and contraction of the implant post-installation.

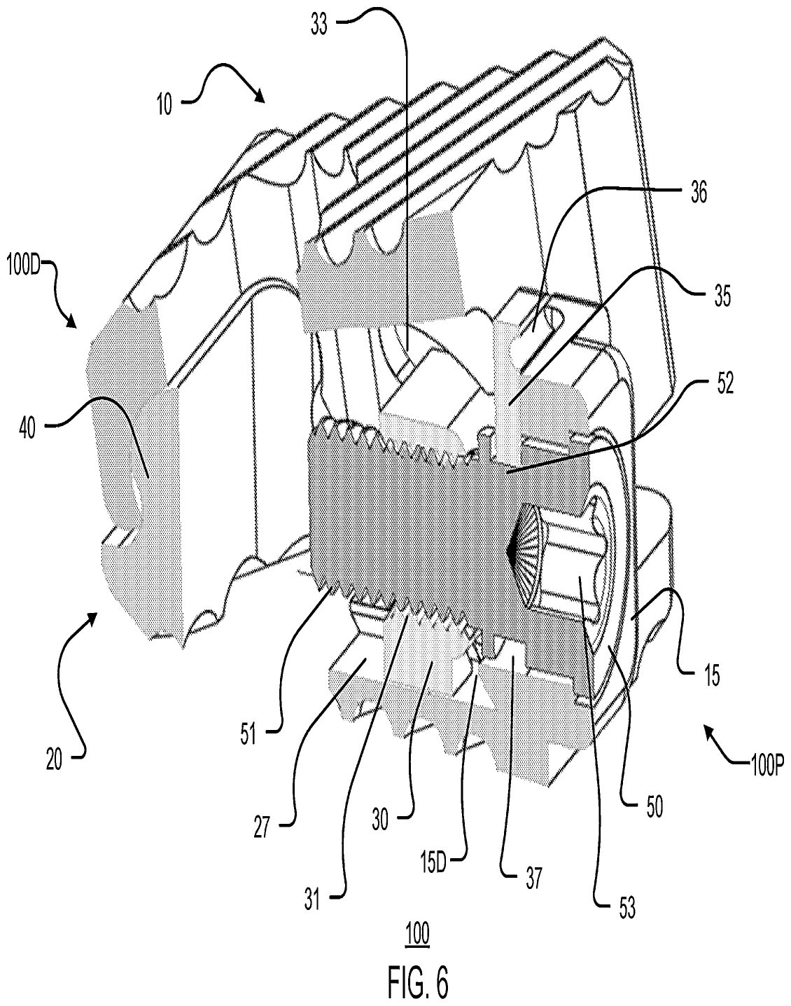

In some embodiments, retaining clip 35 may have a retaining clip groove 36 that faces the proximal direction when seated in recess 37. In various embodiments, a surgeon may lift up on the retaining clip 35 via the clip groove 36 and pull the clip out thereby uncoupling from the expansion screw groove 52. The external thread 51 of expansion screw 50 may have a size and shape generally corresponding to the support frame 15, and threaded portion 31 of drive wedge 30, for example. In various embodiments, drive wedge 30 may include a tang 33 on a lower end which is operatively engaged with and/or coupled with channel 27 on inferior endplate 20 (see also FIG. 6). In this way, the wedge 30 may slide back and forth in the proximal to distal direction P-D and be operatively engaged with the inferior endplate 20. In the example embodiment tang 33 and channel 27 may be operatively coupled by a dovetail configuration although other configurations are contemplated, i.e., tongue and groove etc. In various embodiments, expansion screw 50 may include a drive feature 53 disposed with respect to the proximal end of the expansion screw 50, i.e., the head portion of the expansion screw. In the example embodiment, the drive feature may take a hexalobular shape, although various other shapes such as hexagonal, polygonal shapes having at least three sides, Torx, etc. are also contemplated. In some embodiments, a surgical tool too having a corresponding drive end may be coupled to cause rotation of expansion screw 50 via the drive feature.

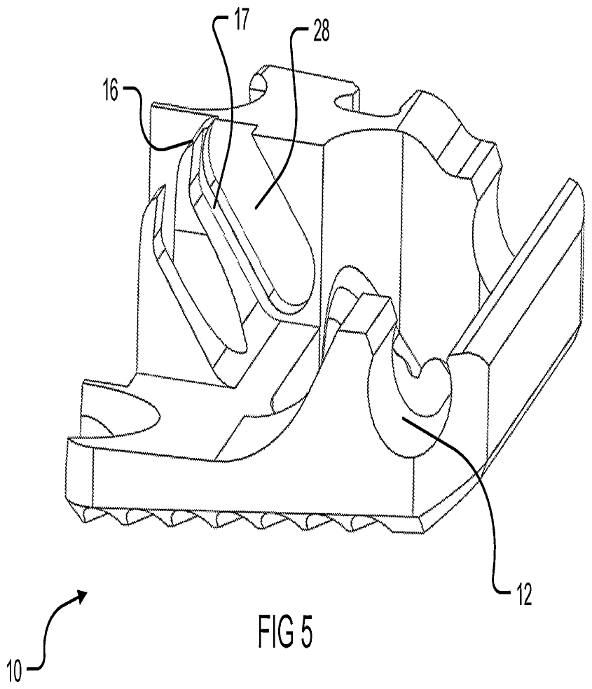

FIG. 5 is a perspective view of the interior surfaces of a superior endplate 10. In the example illustration, it is shown that the distal end of superior endplate 10 includes an arcuate channel 12 extending in the widthwise direction of which the hinge member 40 of the inferior endplate may be disposed inside of. Superior endplate 10 may also include a ramp 16 comprising an inclined surface 17 that is sloped from the distal side of the superior endplate 10 towards the proximal side of the superior endplate 10. In some embodiments, as the expansion screw 50 is rotated and the wedge 30 is translated, the inclined surface 17 of ramp 16 rides on the top of the drive wedge 30. For example, expansion screw 50 may be screwed in the threaded portion 31 of wedge 30 such that rotation thereof may linearly translate drive wedge 30. In the example embodiment, rotation of expansion screw 50 may draw the wedge 30 towards the proximal end 100P of implant 100 thereby pushing the superior endplate 10 upward in the vertical direction and pivoting the implant due to the incline surface 17 of the ramp 16. In various embodiments, wedge pin 32 may extend in a lateral direction from a side surface of wedge 30 and be disposed within wedge pin groove 28 to provide an additional mating feature between wedge 30 and superior endplate 10. In this way, wedge pin 32 may ride upward at an inclined angle that corresponds to an inclined angle of surface 17. For example, wedge pin 32 may ride along groove 28 in a proximal direction to drive the superior endplate 10 upward while allowing superior endplate 10 to pivot around a pivoting axis defined by the connection between hinge member 40 and channel 12. In various embodiments, the cooperation between wedge pin 32 positioned in groove 28 may define the range of angulation of implant 100. In various embodiments, wedge pin 32 and groove 28 are configured to prevent an overexpansion of the implant and to maintain an angular position of the superior endplate 10 relative to the inferior endplate 20. For example, a first end of groove 28 may define a lowest extent of the range of angulation of implant 100 and second end opposite the first end may define a greatest extent of the range of angulation of implant 100. At least one advantage may be to prevent superior endplate 10 from expanding too much.

FIG. 6 is a perspective cross section view of expandable implant 100. In the example embodiment, it is shown that the superior endplate 10 and inferior endplate 20 are coupled together by a hinge member 40, and drive wedge 30 may be positioned behind the proximal face of inferior endplate 20. For example, the drive wedge may be positioned in the internal space of implant 100 behind the support frame 15 of the inferior endplate and within channel 27. Additionally, expansion screw 50 may extend through an aperture in the proximal face of the inferior endplate 20 such that the external thread 51 of the expansion screw 50 may engage with the threaded portion 31 of the drive wedge 30. In various embodiments, the expansion screw 50 engaged with the drive wedge 30 dispose within channel 27 constrains the lateral movement of the superior endplate 100 along W-W axis about hinge member 40 thereby preventing disassembling of the superior endplate 100 from inferior endplate 200. Additionally, retaining clip 35 may be seated in a corresponding recess 37 of the inferior endplate to constrain relative linear movement of the expansion screw 50. In this way, when expansion screw 50 is rotated the expansion screw 50 may remain in place while the wedge 30 is linearly translated towards the proximal end 100P of implant and slides across the inclined surface 17 of ramp 16 thereby separating superior endplate 10 from inferior endplate 20 and expanding implant 100. In the example embodiment, support frame 15 may take a generally rectangular shape and extend from the proximal face of the inferior endplate to form the recess 37 for supporting the clip 35 and to have various gripping portions as will be explained in more detail below.

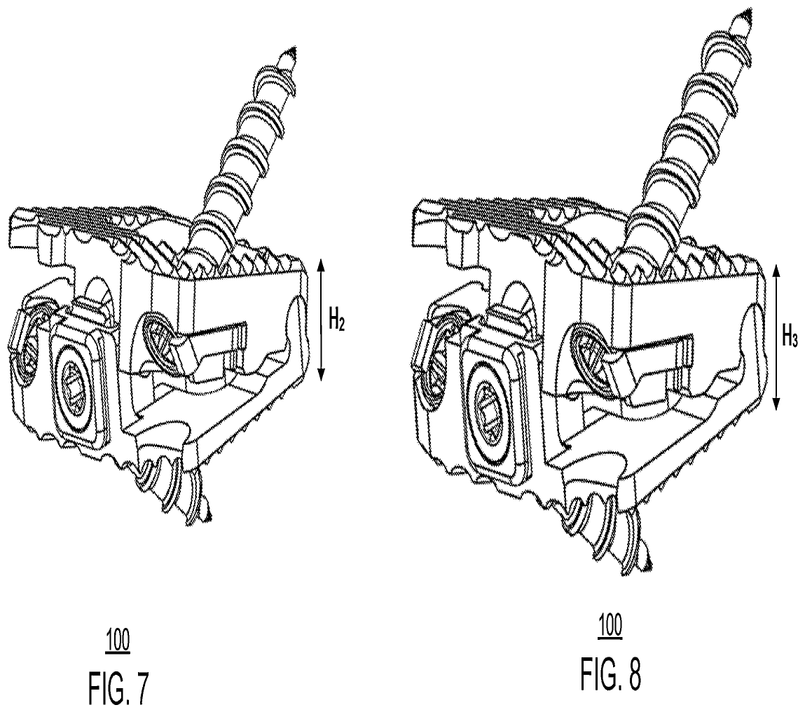

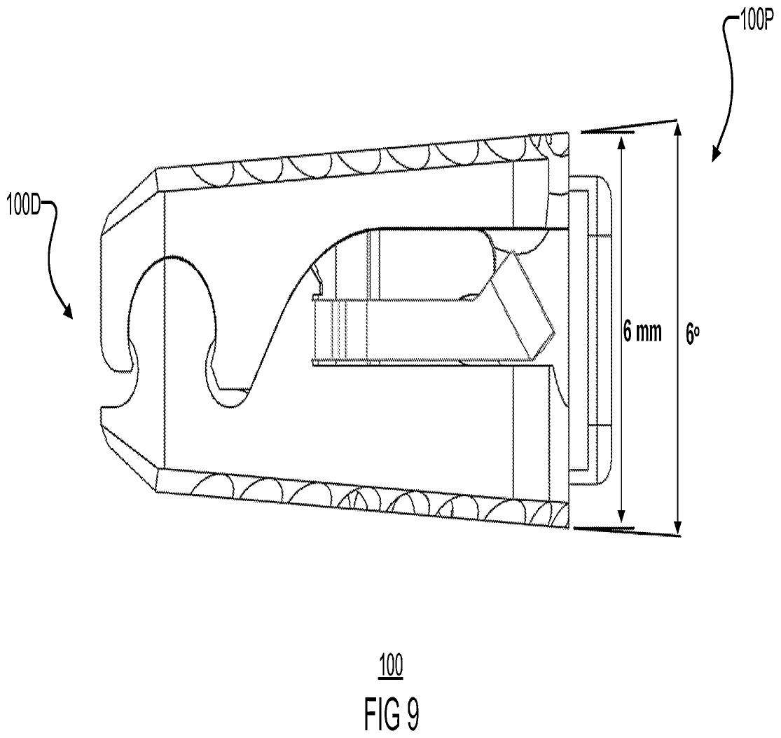

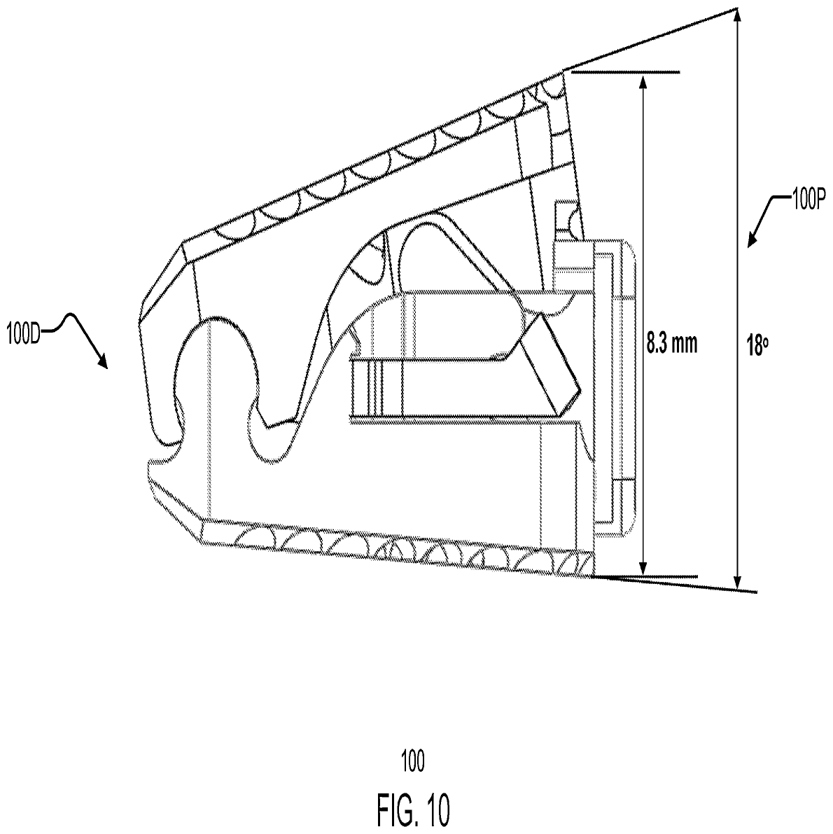

Referring generally to FIGS. 7-10, various views of example embodiments of expandable implant 100 are illustrated. FIG. 7 is a perspective view of a second expandable implant, FIG. 8 is a perspective view of a third expandable implant. In the series of illustrations, it is shown that various embodiments in accordance with the principles of this disclosure may be variously sized in length, width, height, and range of available expansion depending on the particular location in a human body and the particular patient-specific human anatomy. For example, FIG. 7 illustrates a second expandable implant 100 having a second height H₂ (thickness) between the superior endplate 10 and inferior endplate 20 and a second width W₂, FIG. 8 illustrates a third expandable implant 100 having a third height H₃ (thickness) and a third width W₃. In at least some embodiments, H₁ (see FIG. 1) may be about 6 mm, H₂ may be about 7 mm, H₃ may be about 8 mm, for example. In various embodiments, an angle of inclination between the superior endplate 10 and inferior endplate 20 may be about 6 degrees to about 20 degrees in an expanded configuration depending on a size of implant 100, e.g., an angled and/or inclined configuration (FIGS. 9-10).

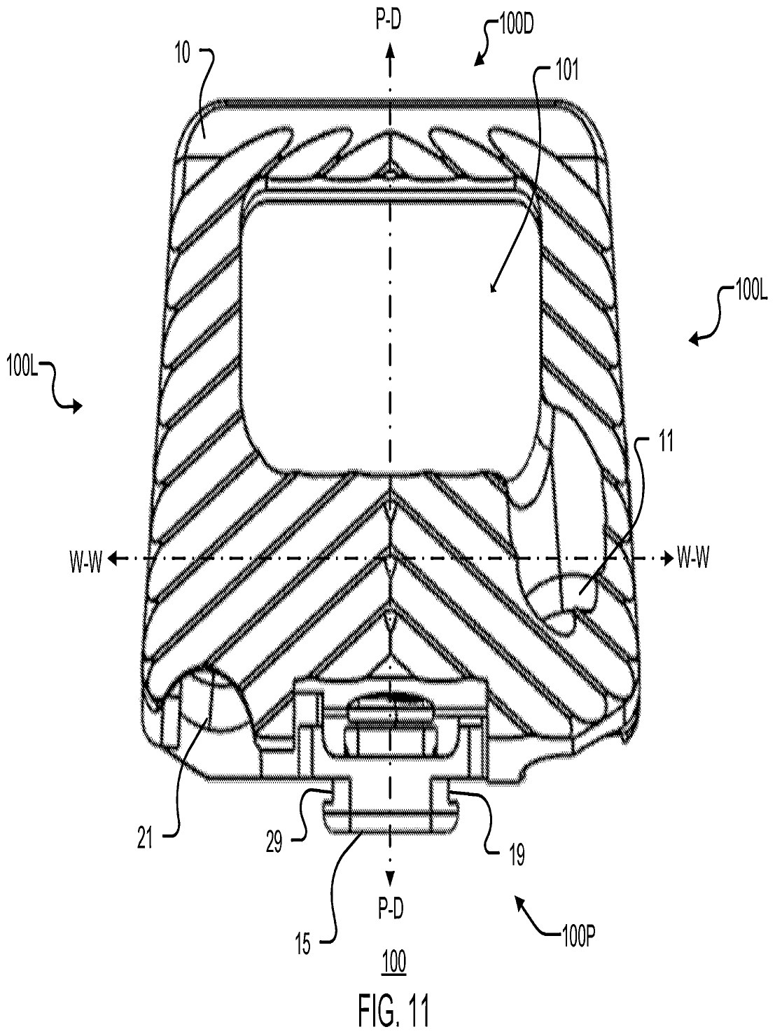

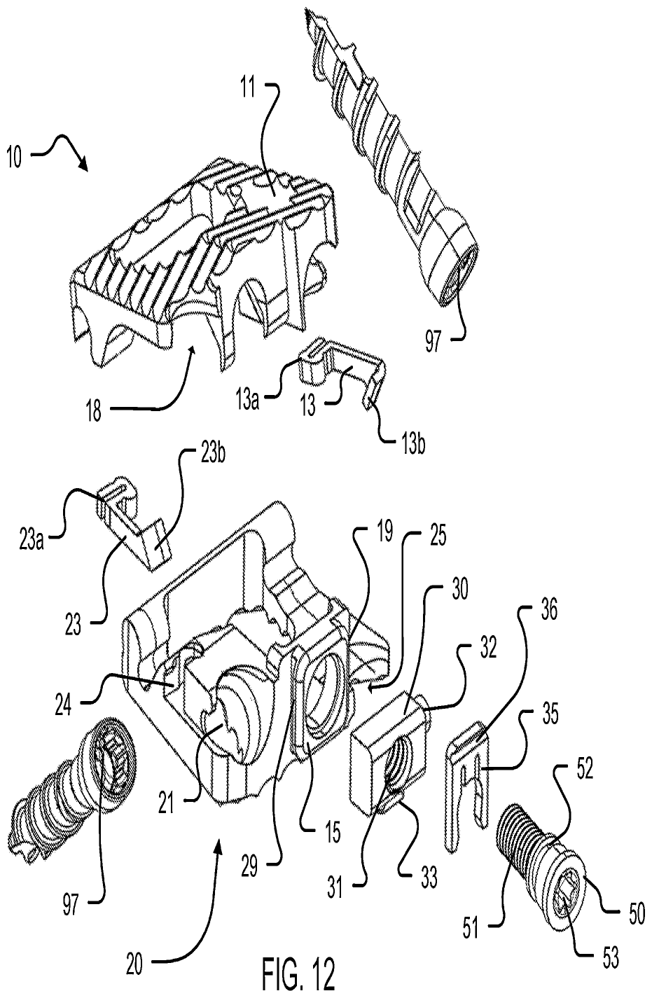

FIG. 11 is a top-down view of another embodiment of an expandable implant 100, and FIG. 12 is a perspective exploded parts view of the expandable implant 100 of FIG. 11. This embodiment may include the same, similar, and/or substantially the same features and components as previously explained. Accordingly, duplicative description regarding the moving mechanism's wedge 30 and expansion screw 50 will be omitted. This embodiment may include bone screw apertures 11, 21 as will be explained in more detail below.

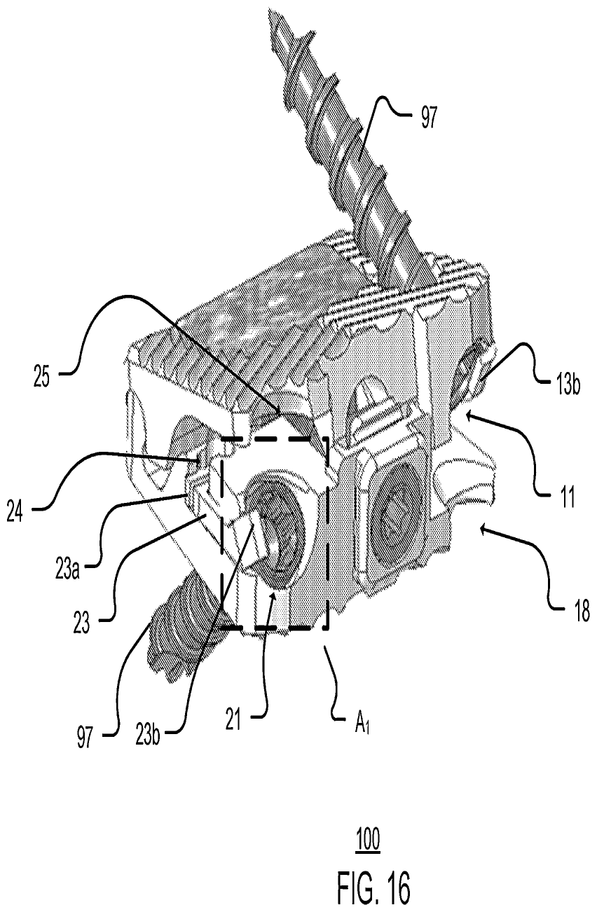

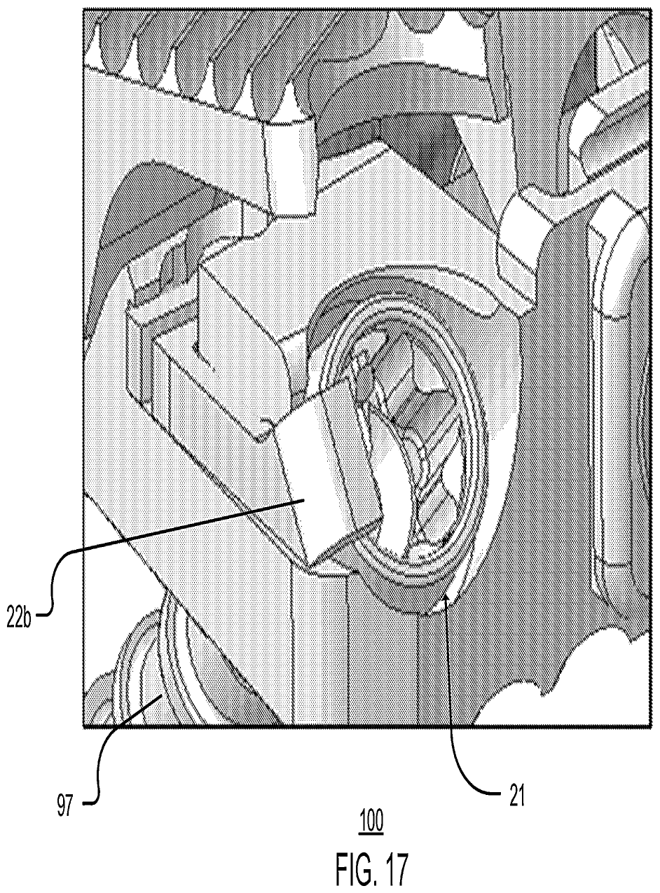

In the example embodiment, a pair of bone screw apertures 11, 21 may be positioned on the proximal end 100P of implant 100. In various embodiments, a bone screw retention mechanism 13, 23 may be provided adjacent the corresponding bone screw aperture 11, 21. In some embodiments, the bone screw retention mechanism 13, 23 may be referred to as an anti-backout locking mechanism. In the example embodiment, the bone screw retention mechanism 13, 23 may comprise a flexible spring like tab member having an anchor portion 13a, 23a at a first end that is anchored into a corresponding cavity 24, 14 of a corresponding endplate. Additionally, the bone screw retention mechanism 13, 23 may include a hook portion 13b, 23b at a second end thereof that has an inclined contact surface. In this way, the bone screw retention mechanism is only anchored to the corresponding endplate at one end thereby allowing it to elastically deform and snap back into place. In operation, the flexible nature of bone screw retention mechanism allows it to flex outward in a lateral direction such that the hook portion moves out of the way seamlessly during the insertion of the bone screw 97 and snaps back over the edge of the head of the bone screw 97 once the bone screw is driven fully into boney anatomy of the patient. For example, as the bone screw 97 is driven in the bone screw aperture 11, 21, the bone screw retention mechanism 13, 23, may automatically flex outward as the head portion of the bone screw contacts the inclined surface of the hook portion 13b, 23b. Stated another way, the bone screw retention mechanism 13, 23 may be configured to move from a neutral position in which the hook portion covers at least a portion of the bone screw aperture to a splayed position in which the hook portion is moved sufficiently far away from the bone screw aperture to allow a bone screw to be driven therethrough the aperture. After driving of the bone screw, the bone screw retention mechanism may snap back to the neutral position and thereby prevent the already driven bone screw from backing out (see FIGS. 16-17).

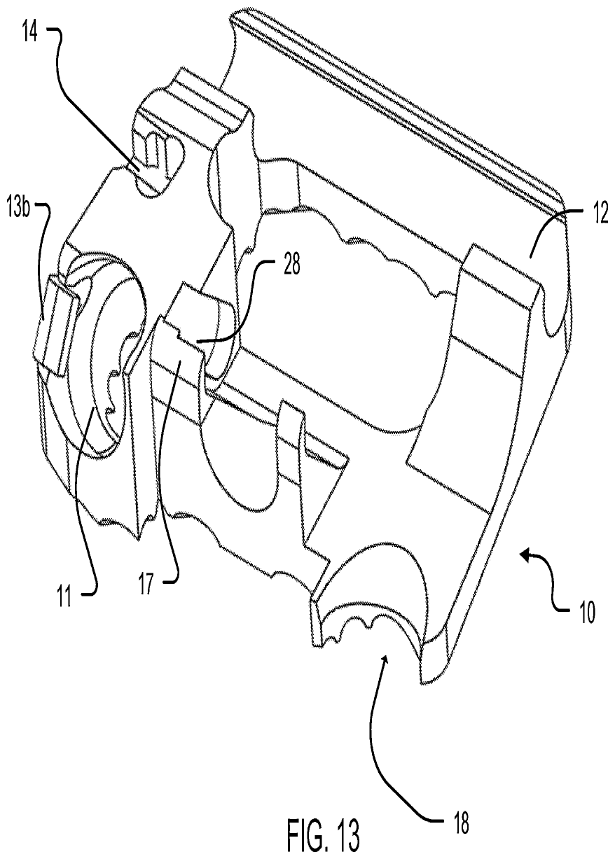

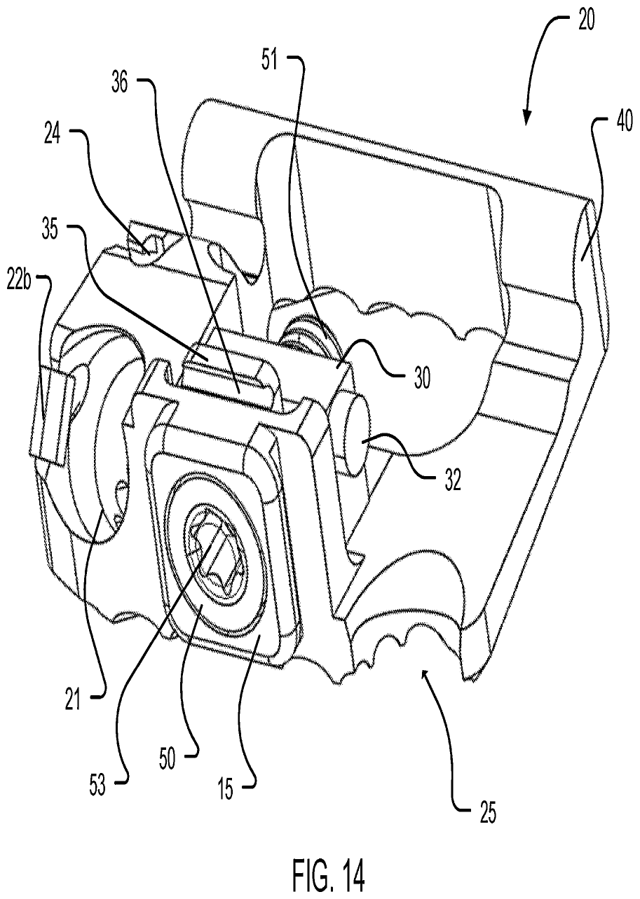

FIG. 13 is a perspective view of the interior surfaces of a superior endplate 10 including a cavity 14 for supporting a bone screw retention mechanism and FIG. 14 is a perspective view of the interior of an inferior endplate 20 including a cavity 24 for supporting a bone screw retention mechanism. The other features of the interior of the superior endplate 10 and inferior endplate 20 may be configured similarly as explained previously. Accordingly, duplicative description will be omitted. The proximal end of superior endplate 10 may include a cutout 18 that functions as relief area for a corresponding bone screw 97 to be inserted through bone screw aperture 21 of inferior endplate 20 without clashing with the superior endplate 10, for example. Similarly, the proximal end of the inferior endplate 20 may include a cutout 25 that functions as a relief area for a corresponding bone screw 97 to be inserted through bone screw aperture 11 of the superior endplate 10 without clashing with the inferior endplate 20, for example.

FIG. 15 is a side view of an expandable implant 100 in the expanded configuration. In an expanded position, an angle of inclination a may be relatively greater in an expanded position than in the closed configuration, for example. In the illustrated expanded configuration of FIG. 15, a may be about 6 degrees to about 20 degrees. In at least one embodiment, implant height may be about 8.3 mm in a fully expanded position and a may be about 18 degrees, for example. In FIG. 15, it is also shown that a centered or bone screw trajectory 99 of bone screw 97 is at an angle β with respect to a transverse plane 98 that crosses through a center of the implant 100 from a first lateral side to a second lateral side, for example. Additionally, the bone screw trajectory 99 may be variable +/− by a degree γ, for example. In this way, a surgeon has some tolerance to install a bone screw +/−γ radially in all directions from the nominal orientation defined by bone screw trajectory 99. In various embodiments, β may be about 30 degrees to about 50 degrees and y may be about 2 degrees to about 10 degrees. In the example embodiment, β may be about 40 degrees and y may be about 5 degrees.

FIG. 16 is a front view of an expandable implant 100 showing an area A1 and FIG. 17 is a front view of the enlarged area A₁ of FIG. 16. In the example embodiment, bone screw 97 is in a position extending through bone screw aperture 21 where it cannot back out due to bone screw retention mechanism 23 as explained above. The bone screw retention mechanism 23 includes an anchor portion 23a anchored in the corresponding cavity 24 inclined surface in the bone screw aperture 21 such that when bone screw 97 is being installed, an underside of the head portion of bone screw 97 directly contacts the inclined surface thereby pushing the hook portion 23b of bone screw retention mechanism 23 laterally outward and away from bone screw aperture 21, for example. Thereafter, when bone screw 97 is installed and the head portion of bone screw 97 is beneath the inclined surface, bone screw retention mechanism 23 may flex back towards bone screw aperture 21 such that it will prevent bone screw 97 from backing out, e.g., a blocking surface of bone screw retention mechanism 23 may contact an upper surface of the head portion of bone screw 97. In the example embodiment, anchor portion 23a may resemble a “c” clip (see FIG. 12) having a bulbous end that may be retaining within cavity 24. Additionally, bone screw retention mechanism 23 comprises a flexible hook (or spring tab) 23b having an inclined surface that is disposed on a lateral end of implant 100 adjacent bone screw aperture 21. The “c” clip shape of anchor portion 23a may be configured to facilitate flexion of the flexible hook 23b.

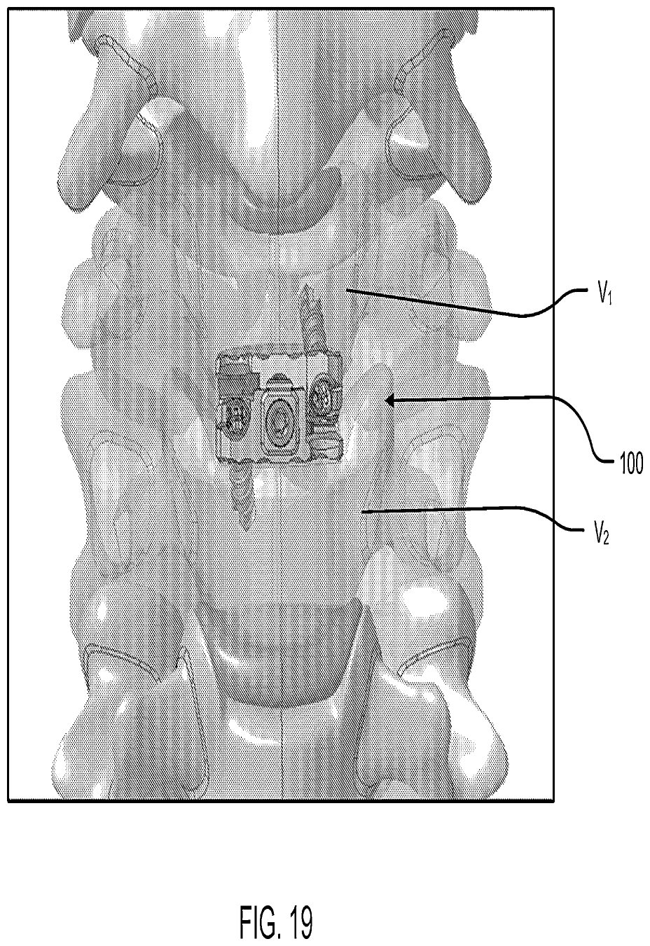

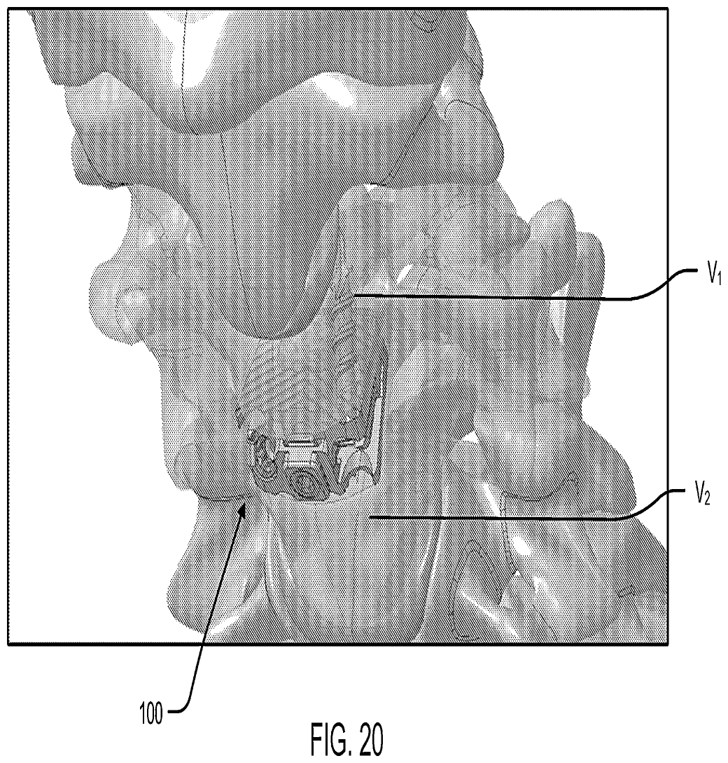

Referring generally to FIGS. 18-20, various views of an expandable implant 100 installed between two vertebrae in an expanded position are illustrated. FIG. 18 is a side perspective view of an embodiment of an expandable implant 100 installed between two vertebrae V₁ and V₂. FIG. 19 is a front view showing the proximal end of an embodiment of an expandable implant 100 installed between two vertebrae V₁ and V₂. FIG. 20 is a perspective view of an embodiment of an expandable implant installed between two vertebrae V₁ and V₂.

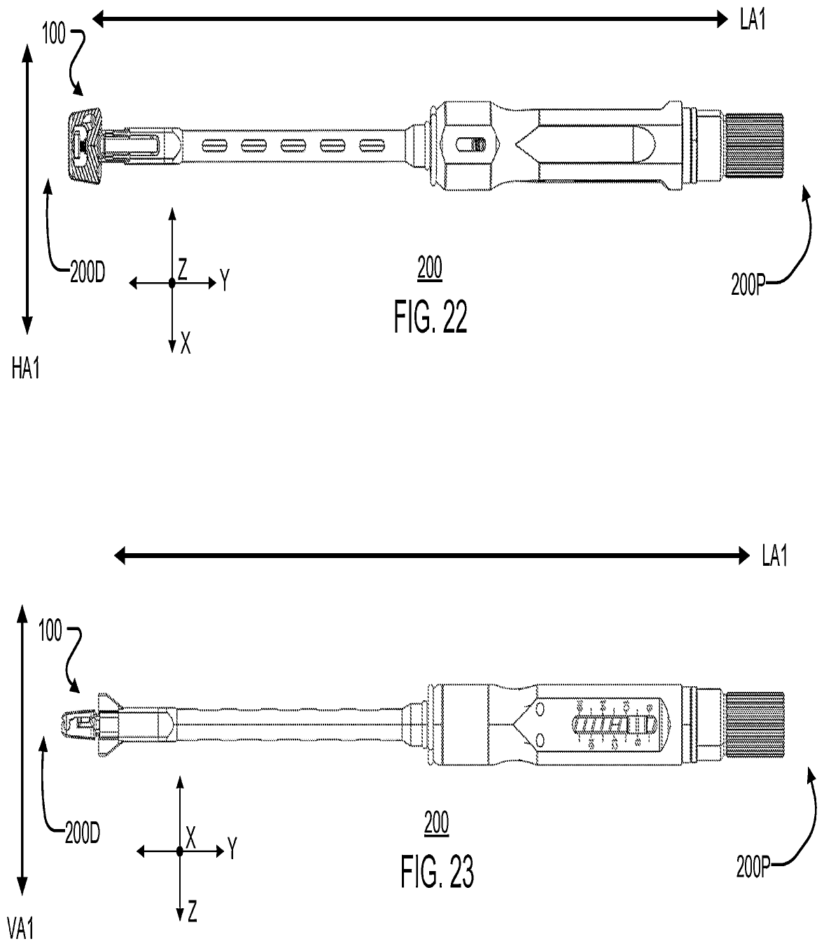

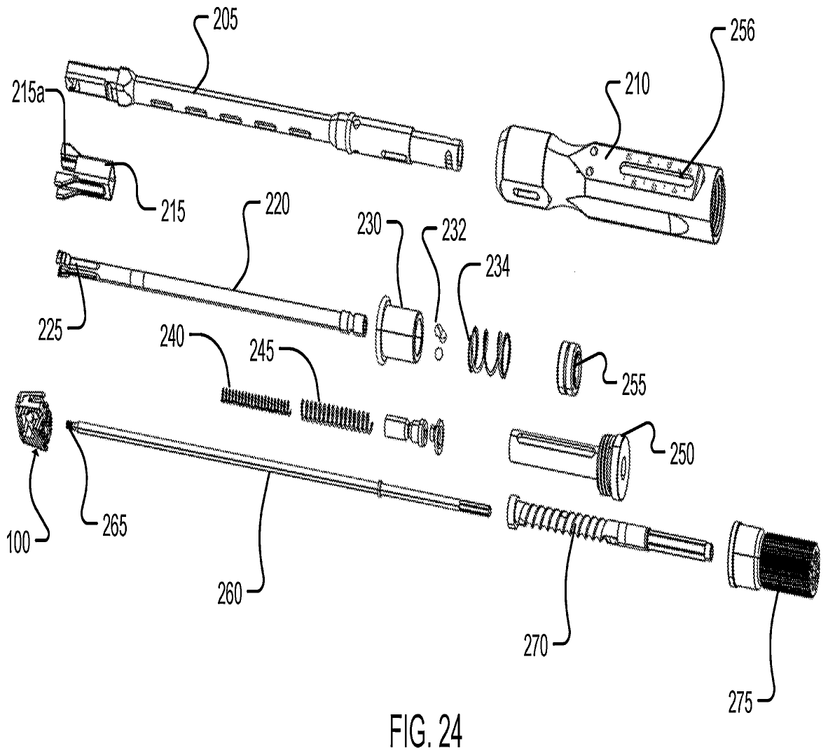

Referring generally to FIGS. 21-24, a surgical tool 200 for use with disclosed expandable implants 100 is illustrated. FIG. 21 is a perspective view of surgical tool 200; FIG. 22 is a top-down perspective view of surgical tool 200; FIG. 23 is a side perspective view of surgical tool 200; and FIG. 24 is exploded parts view. In the example embodiment, surgical tool 200 may extend in a longitudinal direction LA1 from a proximal end 200P to a distal end 200D.

As shown in FIG. 24, the surgical tool 200 may include a jaw shaft assembly for coupling to the implant 100 and a drive shaft assembly for expanding the implant 100. In the example embodiment, an outer body 205 and a handle 210 house and/or support the components of the jaw shaft assembly and the drive shaft assembly. The jaw shaft assembly may comprise a plunger 220, a release actuator 230 for releasing jaw 225, positioning bearings 232, a biasing spring 234, a drive spring 240, and a plunger spring 245. The drive shaft assembly may comprise a drive shaft 260, turn shaft 270, angle indicator ring 255, zero adjustment knob 250, and turn knob 275.

Drive shaft 260 may extend through a hollow, central shaft of the plunger 220. In this way, plunger 220 may be coaxially aligned with and surround the drive shaft 260 and a drive end 265 may extend through the hollow shaft of plunger 220 and position the drive end 265 between the jaw 225. Additionally, the drive shaft 260 may be coupled to turn shaft 270 and may be independently rotatable relative to the plunger 220 by rotating the turn knob 275. For example, drive shaft 260 may be freely rotatable within the central hollow aperture of plunger 220. Additionally, plunger 220 may be independently translated in a longitudinal direction relative to drive shaft 260 by activating actuator 230. In various embodiments, drive shaft 260 is actionable by drive spring 240 and plunger 220 is actionable by plunger spring 245, both disposed at proximal portion of drive shaft 260. Generally, both the drive shaft 260 and plunger 220 will interact with and/or couple implant 100 as will be explained in further detail below.

A mounting jaw 225 for securely coupling to and uncoupling from implant 100 is located at the end of plunger 220 and defines the distal end of surgical tool 200. Mounting jaw 225 provides a latching mechanism for coupling with the gripping grooves 19, 29 of the inferior endplate 20 of implant 100 (see FIG. 25A). Plunger 220 may be disposed inside of outer body 205 and be linearly movable back and forth in the longitudinal direction. For example, a surgeon may slide actuator 230 which translates plunger 220 forward and backward in the longitudinal direction to effectuate horizontal opening and closing of the jaws 225. In some embodiments, surgical tool 200 may include a removable depth stop 215 that is sized to prevent an inadvertent over insertion of the coupled implant 100 into the disc space thereby preventing mounting jaw 225 from releasing implant 100. An additional advantage may be to facilitate the insertion of implant 100 at an appropriate target depth. The jaws 215a of depth stop 215 may be oriented such that a first jaw may contact the caudal vertebrae and the second jaw may contact the cephalad vertebrae. That is, each jaw of depth stop 215a may be perpendicularly oriented with respect to the mounting jaw 225 to control the insertion depth of the implant 100 into the target area.

With reference to the cross-section perspective views of FIGS. 25A and 25B and the exploded parts view of FIG. 24 further details of the coupling process between surgical tool 200 and implant 100 will be disclosed. As seen in FIG. 25B, when actuator 230 is disposed in outer body 205 and is activated against biasing spring 234, the position bearings 235 disposed within the plunger groove 263 release into actuator 230 thereby releasing plunger 220 to move longitudinally in a distal direction by a biasing force applied by plunger spring 245 and splaying mounting jaw 225. In this “open” position, drive shaft end 265 of drive shaft 260 may be disposed within the hollow aperture of plunger 220 and move longitudinally in a distal direct by drive spring 240 to thereby engage with drive feature 53 at the proximal end of expansion screw 50. In various embodiments, drive shaft end 265 may have a size and shape that is designed to conform with the drive feature 53 of expansion screw 50. From the illustrated position of FIG. 25B, the plunger 220 is actuated longitudinally in the distal direction thereby retracting the mounting jaw 225 and engaging with gripping grooves 19, 29 of implant 100, i.e., implant 100 is brought towards surgical tool 200 (see FIG. 25A). In this fully engaged position, drive shaft 260 and plunger 220 are retracted longitudinally in the proximal direction thereby placing position bearings 235 back in plunger groove 263 thereby locking the actuator 230 and preventing inadvertent movement of plunger 220. In this “closed and locked” position, mounting jaw 225 is coupled with gripping grooves 19, 29 and drive end 265 is engaged with drive feature 53 of implant 100 such that implant 100 may be appropriately positioned and expanded.

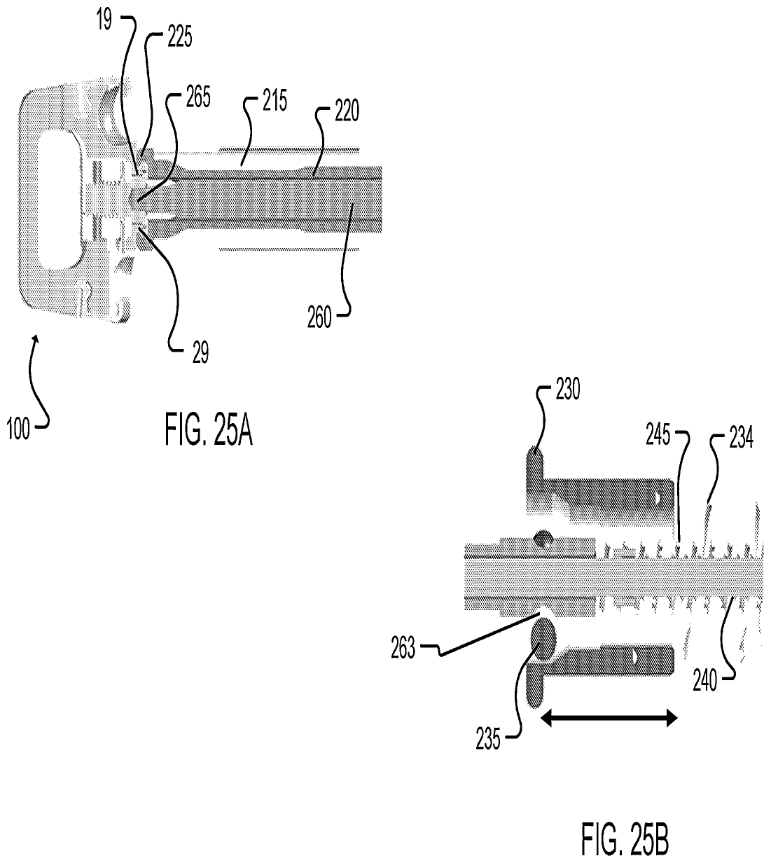

Surgical tool 200 may include a second actuator in the form of a turn knob 275 that is securely coupled to a turn shaft 270. The turn shaft 270 may include a hollow space or aperture therein configured for disposal of drive shaft 260. In this way, turn shaft 270 may be coupled to drive shaft 260 and each may rotate upon actuation of turn knob 275. FIGS. 26A and 26B show the distal and proximal ends of surgical tool 200 with mounting jaw 225 removed for ease of understanding and explanation of an expansion process.

As illustrated in FIG. 26A, once implant 100 is securely mated with surgical tool 200 via drive shaft 260, turn knob 275 may be actionable to rotate along the RI axis (see FIG. 26B) which, in turn, rotates the drive shaft 260 and drive shaft end 265 in the same RI rotation direction (see FIG. 26A). In this way, rotation of turn knob 275 may cause the turn shaft 270 and drive shaft 260 to rotate by an equal amount. In turn, external thread 51 of expansion screw 50 is threadably actuated in the threaded portion 31 thereby linearly translating wedge 30 in the proximal direction along channel 27 which extends along inferior endplate 20 (see FIG. 6). As wedge 30 is translated linearly along channel 27, wedge pin 32 rides along groove 28 and inclined surface 17 of superior endplate 10 thereby separating of superior endplate 10 from inferior endplate 20. For example, superior endplate 10 and inferior endplate 20 may pivotally separate away from one another at hinge member 40, which is disposed within arcuate channel 12.

At the distal end of surgical tool 200, turn knob 275 may be operatively coupled with turn shaft 270 which, in turn, is coupled with an angle indicator ring 255. Angle indicator ring 255 may be used to identify the angle of inclination a of which implant 100 is expanded. Angle indicator ring 255 may be slidable forward and backward along the threads of turn shaft 270 and be disposed inside of handle 210. In this way, rotation of turn knob causes the turn shaft 270 to rotate thereby linearly translating indicator ring 255 back and forth in the longitudinal direction to indicate the angle of inclination. A position of indicator ring 255 is visible through a window 256 in handle 210 which indicates the angle of inclination of the implant. In this way, the surgeon can rotate the expansion screw 50 the appropriate amount such that implant 100 is lordosed to a desired configuration.

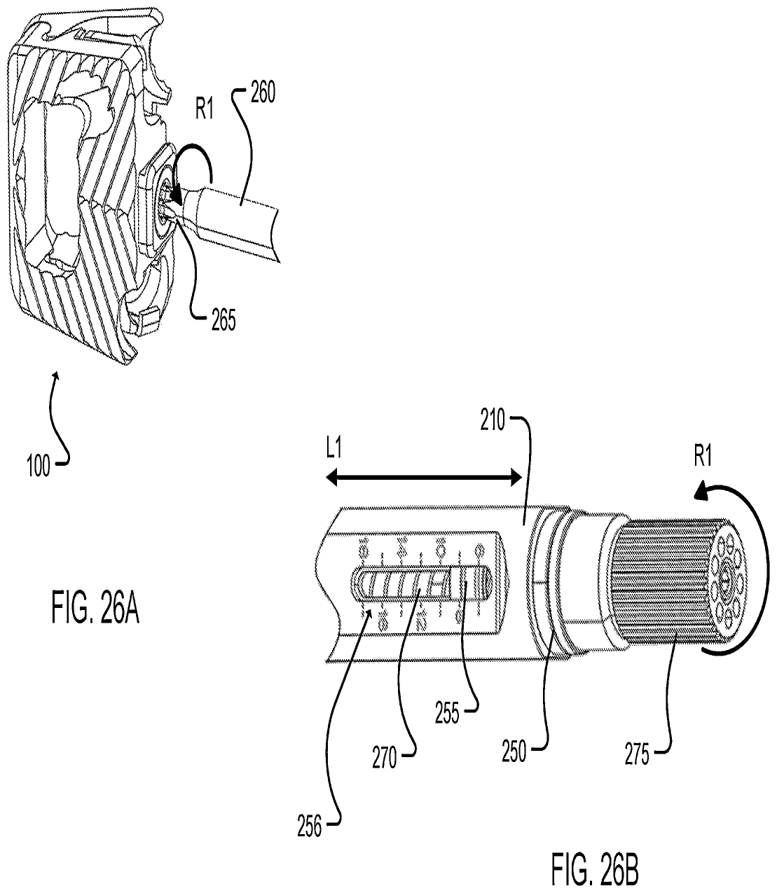

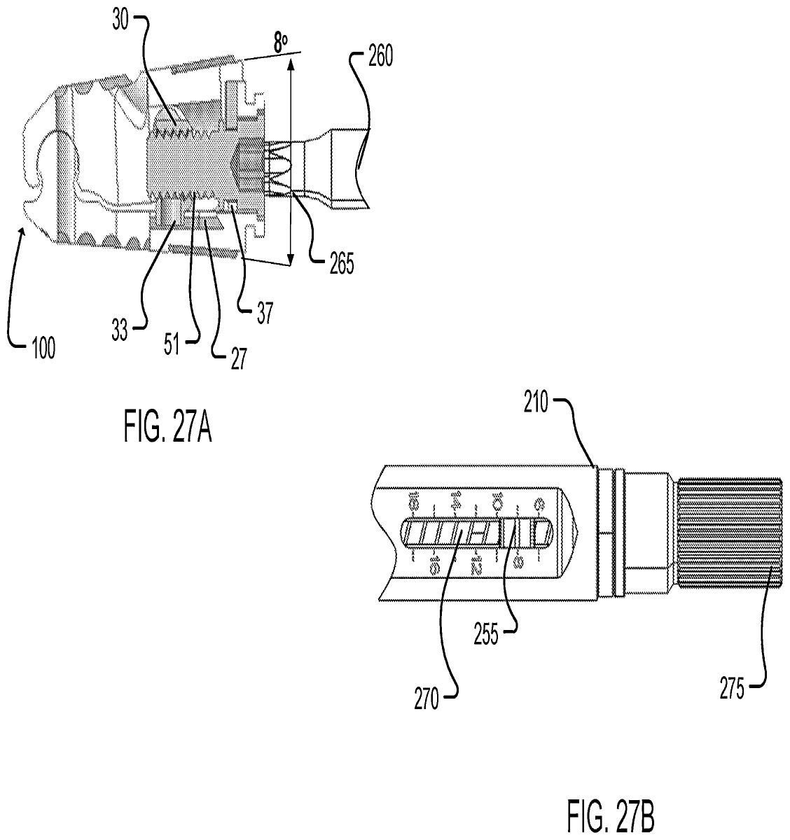

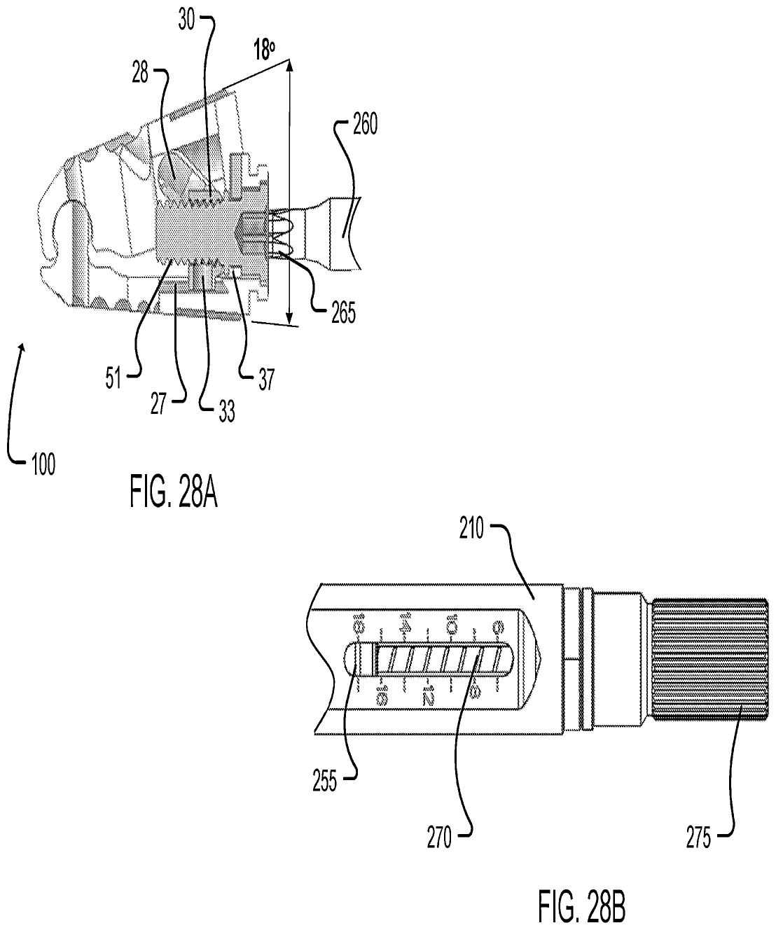

With reference to FIGS. 27A and 27B, drive end 260 of surgical tool 200 is mated to an expandable implant 100 in a closed configuration is disclosed. FIG. 27A shows an implant 100 in a contracted, or a neutral configuration having 8° angle of inclination. Accordingly, the angle indicator 255 coupled to turn shaft 270 disposed in handle 210 shows 8° verifying angle of coupled implant 100 (see FIG. 27B). In the example embodiment in FIGS. 28A and 28B, implant 100 is illustrated in an expanded configuration. Similarly, in this expanded configuration, the angle of inclination of expanded implant 100 is 18° and verified by angle indicator 255. In order to account for implants of varying sizes, surgical tool 200 may be provided with a zero adjustment knob 250 to calibrate the angle indicator ring 255 for differently sized implants. For example, zero adjustment knob 250 may be utilized to calibrate or “zero” the indicator angle for the appropriate implant. In one example, a surgeon would “zero” the indicator angle immediately prior to beginning an expansion process based on a known size and pivoting rate of expansion of implant 100.

A method of operation for expanding an intervertebral implant to a desired angle of inclination will now be discussed. First, a surgeon may couple implant 100 to the surgical tool 200 by operating the actuator 230 thereby splaying open the jaws 225 (see FIGS. 25A, 25B). In this splayed configuration, the exposed drive end 265 may be mated with drive feature 53 of expansion screw 50. Once implant 100 is in the operatively engaged position with drive shaft end 265, mounting jaw 225 may grasp onto gripping grooves 19, 29 for securely engaging surgical tool 200 with implant 100. In this closed configuration, a surgeon may insert implant 100 into the disc space between superior (cephalad) and inferior (caudal) vertebrae, for example. Prior to beginning the expansion process the surgeon may “zero” or calibrate the angle indicator dial by zero adjustment knob 250 as explained above. After the implant 100 is positioned between the superior and inferior vertebrae, a surgeon may rotate turn knob 275 along RI axis rotating the drive shaft 260 and drive shaft end 265 and thereby threadably translating wedge 30 along groove 28 and inclined surface 17 separating the superior endplate 10 apart from the inferior endplate 20. In some embodiments, turn knob 275 may include a torque limiting feature to ensure that expansion screw is tight enough to hold the implant in the expanded configuration but not too tight that shearing might occur.

In various embodiments, a surgeon may verify the expanded angle of implant by the angle indicator ring 255. Once implant 100 is inserted and expanded to the desired configuration, a surgeon may disengage jaw 225 from gripping grooves 19, 29 by activating the actuator 230 to splay the jaw 225 horizontally and releasing from gripping grooves 19, 29. Those with skill in the art will appreciate that the splaying of the jaws horizontally away from one another vs. vertically away from one another is critical to prevent the jaws from being obstructed by the adjacent superior and inferior vertebrae. After disengaging the jaw 225, a surgeon may disengage drive end 265 from drive feature 53 of the expansion screw 50 thereby separating surgical tool 200 from inserted and expanded implant 100.

A method of anchoring or locking an intervertebral implant in a desired angle of inclination within vertebral disc will now be discussed. Once an intervertebral implant is inserted and expanded to a desired angle of inclination in the disc space, a surgeon may lock the superior endplate 10 and inferior endplate 20 in the desired configuration.

At this stage, a surgeon may insert a first bone screw 97 across cutout 25 of inferior endplate 20 and through a first bone screw aperture 11 of superior endplate 10 to operatively anchor second bone screw to superior vertebra. Similarly, a surgeon may insert a second bone screw 97 across cutout 18 of superior endplate 10 and through a second bone screw aperture 21 of inferior endplate 20 to operatively anchor second bone screw 97 to inferior vertebra (FIGS. 13, 14). In some embodiments, a first bone screw retention mechanism 13 is disposed in the corresponding cavity 14 of superior endplate 100 on a first lateral side 100L of implant 100. Similarly, a second bone screw retention mechanism 23 is disposed in the corresponding cavity 24 of inferior endplate 20 on a second lateral side 100L of implant 100. When a surgeon drives bone screw 97 into inclined surface of bone screw aperture 11, 21, hook portion 13b, 23b of bone screw retention mechanism 13, 23 may flex away from the bone screw aperture 11, 21 such that bone screws 97 drives through inclined surface of bone screw aperture 11, 21 and anchors to superior and inferior vertebrae. Thereafter, when bone screw 97 is fully inserted, hook portion 13b, and 23b may flex back towards bone screw aperture 11, 21 such that it will prevent bone crews 97 from backing out fully thereby anchoring implant 100 to superior and inferior vertebrae. In various embodiments, a surgeon may vary bone screw trajectory 99 by +/−γ degrees depending on size and the alignment of cephalad and caudal vertebrae to optimally anchor bone screws 97 to each vertebra (see FIG. 15).

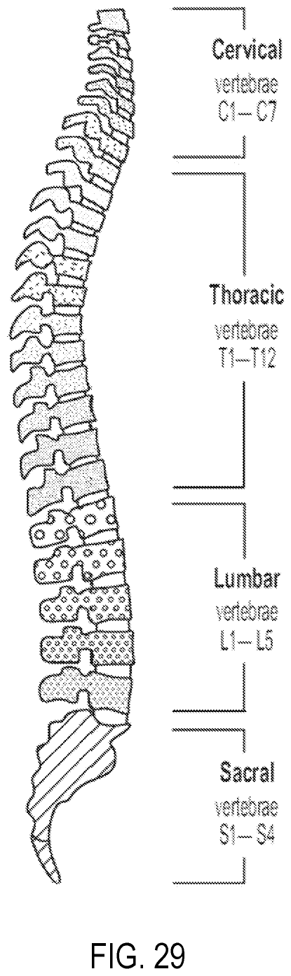

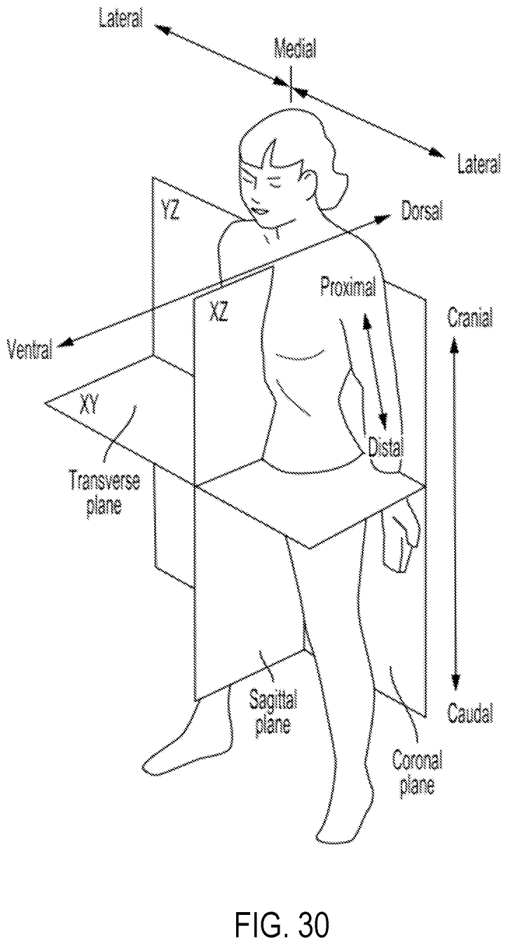

FIG. 29 is a reference drawing showing the human spine of which various disclosed implant embodiments may be installed in. FIG. 30 is a reference drawing showing various planes and reference directions of which the various disclosed implant embodiments may move in or act in with reference to a patient 1.

It should be understood that various aspects disclosed herein may be combined in different combinations than the combinations specifically presented in the description and accompanying drawings. For example, features, functionality, and components from one embodiment may be combined with another embodiment and vice versa unless the context clearly indicates otherwise. Similarly, features, functionality, and components may be omitted unless the context clearly indicates otherwise. It should also be understood that, depending on the example, certain acts or events of any of the processes or methods described herein may be performed in a different sequence, may be added, merged, or left out altogether (e.g., all described acts or events may not be necessary to carry out the techniques).

Unless otherwise specifically defined herein, all terms are to be given their broadest possible interpretation including meanings implied from the specification as well as meanings understood by those skilled in the art and/or as defined in dictionaries, treatises, etc. It must also be noted that, as used in the specification and the appended claims, the singular forms “a,” “an” and “the” include plural referents unless otherwise specified, and that the terms “comprises” and/or “comprising,” when used in this specification, specify the presence of stated features, elements, and/or components, but do not preclude the presence or addition of one or more other features, steps, operations, elements, components, and/or groups thereof.

The techniques of this disclosure may also be described in the following examples.

Example 1: An expandable implant (100) movable between a contracted position and an expanded position, comprising:
a body extending from a proximal end to a distal end in a proximal-to-distal direction and extending from a first lateral side to a second lateral side in a widthwise direction, the expandable body being defined by a superior endplate (10) and an inferior endplate (20) that are pivotally connected; the superior endplate includes a ramp (16) that comprises an inclined surface (17); the inferior endplate comprises support frame (15) inside surface including a channel (27), wherein the support frame includes a frame portion having a proximal surface and a distal surface, wherein an aperture extends through the proximal surface and the distal surface;
a wedge (30) disposed between the superior endplate and the inferior endplate, the wedge including: a bearing surface on an upper end thereof configured to slide across the inclined surface of the ramp and a tang (33) on a lower end thereof configured for disposal within the channel; and
an expansion screw (50) extending through the aperture and being operatively coupled to a threaded portion (31) of the wedge, wherein rotation of the expansion screw is configured to linearly translate the wedge in a longitudinal direction;
wherein proximal movement of the wedge cause the superior endplate to rotate away from the inferior endplate.

Example 2: The expandable implant of example 1, further comprising:
a groove (28) disposed on an interior sidewall of the superior endplate, and
a pin (32) extending from the wedge and being configured to slide within the groove upon linear movement of the wedge,
wherein the pin and the wedge are configured to prevent an overexpansion of the implant and to maintain an angular position of the superior endplate.

Example 3: The expandable implant of example 1, further comprising:
a recess (37) formed within the support frame portion, and
wherein a head portion of the expansion screw resides within the recess.

Example 4: The expandable implant of example 3, further comprising:
a retaining clip (35) seated within the recess, the retaining clip being coupled to a groove portion (52) of the expansion screw, and
wherein the retaining clip is configured to prevent the expansion screw from linearly translating while permitting the expansion screw to rotate; and wherein the retaining clip is configured to provide friction on the expansion screw to prevent the expansion screw from inadvertently rotating after insertion and expansion of the expandable implant.

Example 5: The expandable implant of example 1, further comprising:
at least one bone screw aperture (11, 21) extending through one of the superior endplate or the inferior endplate; and
at least one retention tab (13, 23) having a hook portion (13b, 23b) and an anchor portion (13a, 23a),
wherein the at least one retention tab is configured to automatically deflect away from the at least one bone screw aperture during installation of a corresponding bone screw (97) and return to a retaining position upon full seating of the bone screw within the at least one bone screw aperture.

Example 6: The expandable implant of example 1, further comprising: a first bone screw aperture (11) extending through the superior endplate;
a second bone screw aperture (21) extending through the inferior endplate;
a first retention tab (13) having a first hook (13b) portion and a first anchor portion (13a),
the first retention tab being configured to: (a) cover at least a portion of the first bone screw aperture in a neutral position, and (b) deflect away from the neutral position upon driving of a first bone screw into the first bone screw aperture when a head portion of the first bone screw contacts an inclined surface of the first hook portion; and
a second retention (23) tab having a second hook (23b) portion and a second anchor portion (23a), the second retention tab being configured to: (a) cover at least a portion of the second bone screw aperture in a neutral position, and (b) deflect away from the neutral position upon driving of a second bone screw into the second bone screw aperture when a head portion of the second bone screw contacts an inclined surface of the second hook portion.

Example 7: The expandable implant of example 1, wherein the frame portion further comprises a first gripping groove (19) and a second gripping groove (29).

Example 8: The expandable implant of example 7, wherein:
the first gripping groove comprises a notch on one lateral side of the frame portion; and
the second gripping groove comprises a notch on an opposite lateral side of the frame portion.

Example 9: The expandable implant of example 1, wherein:
the superior endplate comprises a first plurality of engagement features (39) that are angled about 20 degrees to about 40 degrees with respect to a proximal end of the superior endplate; and
the inferior endplate comprises a second plurality of engagement features (39) that are angled about 20 degrees to about 40 degrees with respect to a proximal end of the inferior endplate.

Example 10: The expandable implant of example 1, wherein the channel extends in the proximal-to-distal direction and has a size and shape generally corresponding to a size and shape of the tang to couple the wedge within the channel.

Example 11: The expandable implant of example 1, wherein: the expansion screw comprises a threaded portion (51) at a distal end, a drive feature portion at proximal end (53), and a groove portion connecting the threaded portion and the drive feature portion, and a retaining clip is coupled to the groove portion.

Example 12: The expandable implant of example 1, wherein:
the superior endplate comprises a first bone screw aperture configured to orient a first bone screw in a cephalad direction and a first bone screw relief cutout (18); and
the inferior endplate comprises a second bone screw aperture configured to orient a second bone screw in a caudal direction and a second bone screw relief cutout (25),
wherein the first bone screw relief cutout is configured to provide additional clearance to the second bone screw during an installation process and the second bone screw relief cutout is configured to provide additional clearance to the first bone screw during an installation process.

Example 13: The expandable implant of example 1, further comprising V-shaped engagement features (39).

Example 14: The expandable implant of example 1, further comprising a handle (210) with a window (256).

Example 15: The expandable implant of example 1, further comprising:
a surgical tool (200) for inserting the expandable implant and moving the expandable implant from the contracted position to the expanded position, the surgical tool comprising:
a plunger (220) having a mounting jaw end (225) that is engaged by a jaw release actuator (230);
wherein activating the jaw release actuator moves the mounting jaw to an open configuration greater than a distance between the first gripping groove and the second gripping groove of the frame portion of the expandable implant;
a drive shaft (260) disposed within the plunger having a drive end (265) configured to engage a drive feature of the expansion screw;
wherein the jaw release actuator is configured to engage with the first gripping groove and the second gripping groove by pushing the drive shaft against the expansion screw;
a turn shaft (270) coupled to the drive shaft configured to rotate the drive shaft by actuation of a turn knob handle (275) coupled the turn shaft;
wherein actuating the turn knob handle rotates the drive shaft and the expansion screw to cause the superior endplate to pivotally separate away from the inferior endplate;
an angle indicator ring (275) operatively coupled with the turn shaft,
wherein rotation of the drive shaft translates the angle indicator ring along the turn shaft thereby incrementally identifying a corresponding angle of expansion of the expandable implant throughout rotation of the turn shaft;
a depth stop (215) coupled perpendicularly to the mounting jaw;
wherein the depth stop is configured to engage surrounding vertebrae and prevent over-insertion of the expandable implant thereby preventing the mounting jaw from releasing the implant;
an outer body (205) extending through the plunger and the drive shaft; and
a handle (210) enclosing the turn shaft and the angle indicator ring.

Various examples of the disclosure have been described. These and other examples are within the scope of the following claims.

What is claimed is:

1. An expandable implant movable between a contracted position and an expanded position, comprising:
a body extending from a proximal end to a distal end in a proximal-to-distal direction and extending from a first lateral side to a second lateral side in a widthwise direction, the body being defined by a superior endplate and an inferior endplate that are pivotally connected;
wherein the superior endplate includes an inside surface that comprises an inclined ramp;
wherein the inferior endplate comprises a support frame, an inside surface including a channel, wherein the support frame includes a frame portion having a proximal surface and a distal surface, wherein an aperture extends through the proximal surface and the distal surface;
a wedge disposed between the superior endplate and the inferior endplate, the wedge including: a bearing surface on an upper end thereof configured to slide across the inclined ramp and a tang on a lower end thereof configured for disposal within the channel; and
an expansion screw extending through the aperture and being operatively coupled to a threaded portion of the wedge, wherein the expansion screw is configured to rotate and thereby linearly translate the wedge in a longitudinal direction;
wherein proximal movement of the wedge causes the superior endplate to rotate away from the inferior endplate.

2. The expandable implant of claim 1, further comprising:
a groove disposed on an interior sidewall of the superior endplate; and
a pin extending from the wedge and being configured to slide within the groove upon linear movement of the wedge,
wherein the pin and the wedge are configured to prevent an overexpansion of the implant and to maintain an angular position of the superior endplate relative to the inferior endplate.

3. The expandable implant of claim 1, further comprising:
a recess formed within the support frame portion, and
wherein a head portion of the expansion screw resides within the recess.

4. The expandable implant of claim 3, further comprising:
a retaining clip seated within the recess, the retaining clip being coupled to a groove portion of the expansion screw;
wherein the retaining clip is configured to prevent the expansion screw from linearly translating while permitting the expansion screw to rotate; and
wherein the retaining clip is configured to provide friction on the expansion screw to prevent the expansion screw from inadvertently rotating after insertion and expansion of the expandable implant.

5. The expandable implant of claim 1, further comprising:
at least one bone screw aperture extending through one of the superior endplate or the inferior endplate; and
at least one retention tab having a hook portion and an anchor portion;
wherein the at least one retention tab is configured to automatically deflect away from the at least one bone screw aperture during installation of a corresponding bone screw and return to a retaining position upon full seating of the bone screw within the at least one bone screw aperture.

6. The expandable implant of claim 1, further comprising:
a first bone screw aperture extending through the superior endplate;
a second bone screw aperture extending through the inferior endplate;
a first retention tab having a first hook portion and a first anchor portion, the first retention tab being configured to: (a) cover at least a portion of the first bone screw aperture in a neutral position, and (b) deflect away from the neutral position upon driving of a first bone screw into the first bone screw aperture when a head portion of the first bone screw contacts an inclined surface of the first hook portion; and
a second retention tab having a second hook portion and a second anchor portion, the second retention tab being configured to: (a) cover at least a portion of the second bone screw aperture in a neutral position, and (b) deflect away from the neutral position upon driving of a second bone screw into the second bone screw aperture when a head portion of the second bone screw contacts an inclined surface of the second hook portion.

7. The expandable implant of claim 1, wherein the frame portion further comprises a first gripping groove and a second gripping groove.

8. The expandable implant of claim 7, wherein:
the first gripping groove comprises a notch on one lateral side of the frame portion; and
the second gripping groove comprises a notch on an opposite lateral side of the frame portion.

9. The expandable implant of claim 1, wherein:
the superior endplate comprises a first plurality of engagement features that are angled about 20 degrees to about 40 degrees with respect to a proximal end of the superior endplate; and
the inferior endplate comprises a second plurality of engagement features that are angled about 20 degrees to about 40 degrees with respect to a proximal end of the inferior endplate.

10. The expandable implant of claim 1, wherein the channel extends in the proximal-to-distal direction and has a size and shape generally corresponding to a size and shape of the tang to couple the wedge within the channel.

11. The expandable implant of claim 1, wherein: the expansion screw comprises a threaded portion at a distal end, a drive feature portion at proximal end, and a groove portion connecting the threaded portion and the drive feature portion, and a retaining clip is coupled to the groove portion.

12. The expandable implant of claim 1, wherein:
the superior endplate comprises a first bone screw aperture configured to orient a first bone screw in a cephalad direction and a first bone screw relief cutout; and
the inferior endplate comprises a second bone screw aperture configured to orient a second bone screw in a caudal direction and a second bone screw relief cutout;
wherein the first bone screw relief cutout is configured to provide additional clearance to the second bone screw during an installation process and the second bone screw relief cutout is configured to provide additional clearance to the first bone screw during an installation process.

13. A system including a medical implant and a surgical tool, the system comprising:
an expandable implant movable between a contracted position and an expanded position, comprising:
a body extending from a proximal end to a distal end in a proximal-to-distal direction and extending from a first lateral side to a second lateral side in a widthwise direction, the expandable body being defined by a superior endplate and an inferior endplate that are pivotally connected;
the superior endplate includes an inside surface that comprises an inclined ramp;
the inferior endplate comprises a support frame, an inside surface including a channel, and a frame portion having a proximal surface and a distal surface, wherein an aperture extends through the proximal surface and the distal surface of the frame portion;
a wedge disposed between the superior endplate and the inferior endplate, the wedge including: a bearing surface on an upper end thereof configured to slide across the inclined ramp and a tang on a lower end thereof configured for disposal within the channel; and
an expansion screw extending through the aperture and being operatively coupled to the wedge;
wherein the expansion screw is configured to rotate and thereby linearly translate the wedge in a longitudinal direction; and
a surgical tool for moving the expandable implant from the contracted position to the expanded position;
wherein rotation of a driver of the surgical tool causes the wedge to move proximally and causes the superior endplate to rotate away from the inferior endplate.

14. The system of claim 13, wherein:
the inferior endplate comprises a first gripping groove located at a first lateral side of the frame portion, and a second gripping groove located at a second lateral side of the frame portion;
the surgical tool comprises a mounting jaw configured to engage with the first gripping groove and the second gripping groove; and
wherein in an insertion configuration, the first gripping groove and the second gripping groove of the expandable implant are engaged by the mounting jaw of the surgical tool.

15. The system of claim 14, wherein the surgical tool comprises:
a plunger having a jaw actuator configured to cause the mounting jaw to affirmatively engage and disengage with the gripping grooves of the expandable implant;
wherein activating the jaw actuator moves the mounting jaw to an open configuration by a distance that is greater than a distance between the first gripping groove and the second gripping groove of the frame portion of the expandable implant;
a drive shaft disposed within the plunger having a drive end configured to engage a drive feature of the expansion screw;
wherein the jaw actuator is configured to engage with the first gripping groove and the second gripping grove by pushing the drive shaft against the expansion screw;
a turn shaft coupled to the drive shaft and being configured to rotate the drive shaft by actuation of a turn knob coupled to the turn shaft;
wherein actuating the turn knob rotates the drive shaft and the expansion screw to cause the superior endplate to pivotally separate away from the inferior endplate;
an angle indicator ring operatively coupled with the turn shaft;
wherein rotation of the drive shaft translates the angle indicator ring along the turn shaft thereby incrementally identifying a corresponding angle of expansion of the expandable implant throughout rotation of the turn shaft;
an outer body surrounding the plunger and the drive shaft; and
a handle enclosing the turn shaft and the angle indicator ring.

16. The system of claim 15, wherein rotation of the expansion screw causes the wedge disposed in the channel to slide within the inferior endplate towards the proximal end of the expandable implant thereby causing the bearing surface of the wedge to engage the inclined ramp to separate the superior endplate from the inferior endplate along a pivoting axis.

17. The system of claim 16 further comprising a depth stop coupled perpendicularly to the mounting jaw, the depth stop configured to engage surrounding vertebrae and prevent over-insertion of the expandable implant thereby preventing the mounting jaw from releasing the implant.

18. The system of claim 17, wherein an expanded angle between the superior endplate from the inferior endplate is identified by indicator ring of the surgical tool.

19. The system of claim 18, further comprising a retaining clip engaged in a recess of the frame portion, wherein the retaining clip is configured to engage a corresponding slot of the expansion screw to prevent the expansion screw from linearly translating while permitting the expansion screw to be rotated by a driver.

20. The system of claim 19, wherein the retaining clip is configured to provide friction on the expansion screw to prevent the expansion screw from inadvertently rotating in absence of the driver preventing unwanted expansion and contraction of the implant after insertion and expansion.
